(19) United States
(12) Patent Application Publication
(10) Pub. No.: US 2024/0342343 A1
(43) Pub. Date: Oct. 17, 2024

(54) OPEN TYPE IMPLANTABLE CELL DELIVERY DEVICE

(71) Applicants: UNIVERSITEIT MAASTRICHT, MAASTRICHT (NL); ACADEMISCH ZIEKENHUIS MAASTRICHT, MAASTRICHT (NL)

(72) Inventors: Aart Alexander VAN APELDOORN, MAASTRICHT (NL); Rick DE VRIES, MAASTRICHT (NL)

(21) Appl. No.: 18/682,652

(22) PCT Filed: May 18, 2022
(86) PCT No.: PCT/EP2022/063424

§ 371 Date: Feb. 9, 2024

(30) Foreign Application Priority Data
Aug. 10, 2021 (EP) 21190655.7

(51) Int. Cl. A61L 27/56; A61L 27/16; A61L 27/38; A61L 27/52; A61L 27/54; B32B 3/02; B32B 3/26; B32B 3/30; B32B 7/05; B32B 27/08; B32B 27/30; B32B 27/36

(52) U.S. Cl. CPC A61L 27/56; A61L 27/16; A61L 27/3804; A61L 27/52; A61L 27/54; B32B 3/02; B32B 3/26; B32B 3/30; B32B 7/05; B32B 27/08; B32B 27/304; B32B 27/36; B29K 2995/0056; B32B 2250/24; B32B 2255/10; B32B 2535/00

(57) ABSTRACT

An open type implantable cell delivery device for transplanting cells in a subject, comprising: a bottom film having a surface area with a plurality of pores; a top film having a surface area with a plurality of pores, positioned on top of the bottom film such that the top film substantially covers the bottom film to create an inner space; wherein the bottom film and the top film are formed from a biocompatible biomaterial, and wherein the bottom film comprises a plurality of microwells positioned to face the surface area of the top film with the open sides of said microwells, wherein the pore size of the bottom film and optionally the top film is such that it allows vascularization or vascular ingrowth in the device through the pores.

### FIELD OF THE INVENTION

The invention relates to the field of implantable cell delivery devices, particularly implantable cell delivery devices of the open type, which allow interconnection of implanted cells and host tissue. Such devices may be used for transplanting cells such as pancreatic islet cells in a subject. The invention further relates to a method for constructing the device and a method of loading the device with cells. Further, the invention is related to the use of the open type implantable cell delivery device containing cells in the treatment of a disease or disorder by implanting the device in a subject.

### BACKGROUND

Transplantation of donor cells in patient holds a promising tool for the treatment of a variety of diseases. For example, islet cells may be transplanted in diabetes patients. Currently, clinical islet transplantation is the most promising minimal invasive therapy to treat the most severe cases of type 1 diabetes in which exogenous insulin administration can no longer be used to control blood glucose levels. During this procedure, the pancreas of a deceased donor is harvested, pancreatic islets are isolated and subsequently transplanted in a type 1 diabetic patient. The pancreas itself is not considered as a suitable transplantation site for pancreatic islets, due to the possible leakage of digestive enzymes and the high risk of pancreatitis. Therefore, hepatic delivery of islets through the portal vein has been the golden standard for clinical islet transplantation. Despite great progress in isolation and transplantation protocols in the last two decades, less than 40% of patients show insulin independence 5 years after islet transplantation, which further reduces to 30% after 10 years. Furthermore, pancreatic islet transplantation is associated with a loss of 60% of transplanted islets within hours post transplantation, which explains the need for an average of 2-3 donors to cure one type 1 diabetic patient. This decrease of islet mass over time is caused by, amongst others, mechanical stress, a lack of oxygen flow to the islets due to impaired vascularization and the presence of an immediate blood-mediated immune response within the liver. The oxygen tension of islets within the pancreas is reported to be 30-40 mm Hg, which can increase close to the oxygen tension of arterial blood (80-100 mm Hg) since islets in the pancreas contain a dense capillary network. Transplanted islets are known to revascularize in roughly 14 days, but even after 3 months, intrahepatic transplanted islets show a relative low oxygen tension (<10 mmHg). In addition, commonly used immunosuppressive drugs are taken orally, which have a first hepatic passage with the highest drug levels to be found in the liver. This can contribute to islet injury as the immunosuppressive drugs have shown to be toxic to islets. Offering pancreatic islets an extra-hepatic transplantation site through the help of a macro-encapsulating implant (implantable cell delivery device) is assumed to improve transplantation success.

There are two types of macro-encapsulating implantable cell delivery devices: one being ‘closed’ immunoprotective devices where macromolecules can enter and exit the device, but cells cannot. For example by controlling the device's pore size to be below 0.45 micron. The fabrication of functional closed (immunoprotective) implantable cell delivery devices remains challenging as the small pore sizes required for blocking immune cells also limit the diffusion of nutrients and for example insulin. The other group consists of ‘open’ devices which allows cells to enter and exit the device, especially aimed to stimulate islet revascularization.

Open implantable cell delivery devices currently on the market or being tested, such as the VC-02 or PEC-Direct device from ViaCyte [A safety, Tolerability, and Efficacy Study of VC-02 Combination Products in Subjects With Type 1 Diabetes Mellitus and Hypoglycemia Unawareness. https://clinicaltrials.gov/ct2/show/NCT03163511] The open nature of the device stimulates swift revascularization of the cells upon implantation. Although the use of immunosuppressive drugs may still be used after implantation of such an open implant, the cells can be transplanted in a less hostile environment. Furthermore, immunosuppressive drugs may not be necessary when cells are engineered to evade the immune response, something which is currently being developed within the field. This could potentially lead to a reduction in the amount of donor organs needed for transplantation and, in the case of transplantation of islet cells, could offer a better maintenance of glycemia, reduction or absence of the need for exogenous insulin injections and lower the risk of long-term complications.

A drawback of existing open type devices is that the cells tend to aggregate. Aggregation of cells tends to cause necrosis of the cells at the center of the cell mass due to deprivation of nutrients and oxygen. A way to prevent aggregation is embedding the cells (or cell clusters) in a hydrogel. However the drawback of this solution is that embedding in hydrogels again hinders diffusion of nutrients and proteins, thus partly undoing the advantages of the open type device. Therefore, improved open type devices are needed.

The inventors previously reported an open microwell-array islet delivery strategy that was successfully used to reverse chemically-induced diabetes in mice (Buitinga, M., et al., Microwell scaffolds for the extrahepatic transplantation of islets of Langerhans. PLoS One, 2013. 8(5): p. e64772; Buitinga, M., et al., Micro-fabricated scaffolds lead to efficient remission of diabetes in mice. Biomaterials, 2017. 135: p. 10-22; both references hereby incorporated by reference in its entirety). The device described herein addresses some of the above problems. The device consists of two thin, porous polymer films. One film is imprinted with a dense array of microwells, a feature unique to this islet delivery device. The other film acts as a lid, entrapping the islets seeded within the microwells. Overall, the device provides physical protection to the islets while the pores in the sheets enable revascularization. The microwell structure ensures that individual islets can be captured in each microwell, leading to a uniform distribution of islets throughout the device and prevention of islet aggregation, reducing the loss of islet cell viability.

The microwell-array implantable cell delivery device however has a few disadvantages. The device is constructed from a specific PolyActive™ composition, which has not been approved for clinical use. Recently, concerns have been raised that this material may induce necrosis in cells, which is undesirable for a cell delivery device. An additional disadvantage is that the microwell-patterned film is closed by suturing a lid on top, which is cumbersome and results in a relatively fragile device. Moreover, the device merely consisted of two thin membranes, which lack mechanical stability, allowing folding of the device. Lastly, enlarging the device towards clinically relevant dimensions for human patients would lead to device dimensions that are surgically challenging to implant.

The present invention addresses the above problems, among others, by the open type device as defined in the appended claims.

### SUMMARY OF THE INVENTION

In a first aspect, the invention relates to an open type implantable cell delivery device for transplanting cells in a subject, comprising:
a bottom film having a surface area with a plurality of pores;
a top film having a surface area with a plurality of pores, positioned on top of the bottom film such that the top film substantially covers the bottom film to create an inner space;
   wherein the bottom film and the top film are formed from biocompatible biomaterial,
   wherein the bottom film comprises a plurality of microwells positioned to face the surface area of the top film with the open sides of said microwells, and
   wherein the pore size of the bottom film and optionally the top film is such that it allows vascularization or vascular ingrowth in the device through the pores.

In a second aspect, the invention relates to the open type implantable cell delivery device according to the first aspect of the invention for use in the treatment, prevention or amelioration of a disease.

In a third aspect, the invention relates to a method of constructing an open type implantable cell delivery device, the method comprising: providing a bottom film having a surface area with a plurality of pores and further comprising a plurality of microwells; positioning a top film having a surface area with a plurality of pores on the bottom film such that the openings of the microwells face the top film, to create an inner space between the bottom and top film in open contact with the microwells; and optionally, positioning a support structure substantially around the assembly of bottom and top films in the same plane as the films such that the support structure at least partly overlaps with the edges of bottom and the top films; spot welding the bottom and the top films in two or more places to attach the bottom and top film to each other and/or to the support structure such as to leave several openings through which the inner space is accessible, and wherein the pore size of the bottom film and optionally the top film is such that it allows vascularization or vascular ingrowth in the device through the pores.

In a fourth aspect the invention relates to a method of seeding an open type implantable cell delivery device as defined in the first aspect of the invention or obtained or obtainable by the method according to the third aspect of the invention with cells, the method comprising: connecting a container for cells with a first end of a tube, and inserting the second end of the tube through an opening of the open type implantable cell delivery device into the inner space such that the inner space is in open connection with the container; clamping the exterior of the open type implantable cell delivery device such that all remaining openings are sealed; loading the container with cells suspended in a suitable medium; allowing the cells to flow from the container through the tube into the inner space of the open type implantable cell delivery device while excess medium is drained through the pores.

### Definitions

For purposes of the present invention, the following terms are defined below.

As used herein, the singular form terms “A,” “an,” and “the” include plural referents unless the content clearly dictates otherwise. Thus, for example, reference to “a cell” includes a combination of two or more cells, and the like.

As used herein, the term “and/or” refers to a situation wherein one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein, the term “conventional techniques” refers to a situation wherein the methods of carrying out the conventional techniques used in methods of the invention will be evident to the skilled worker. The practice of conventional techniques in molecular biology, biochemistry, computational chemistry, cell culture, tissue engineering, regenerative medicine, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

As used herein, the term “in vitro” refers to experimentation or measurements conducted using components of an organism that have been isolated from their natural conditions.

As used herein, the term “subject” or “individual” or “animal” or “patient” or “mammal,” used interchangeably, refer to any subject, particularly a mammalian subject, for whom diagnosis, prognosis, or therapy is desired. Mammalian subjects include humans, domestic animals, farm animals, and zoo-, sports-, or pet-animals such as dogs, cats, guinea pigs, rabbits, rats, mice, horses, cattle, cows, bears, and so on. As defined herein a subject may be alive or dead. Samples can be taken from a subject post-mortem, i.e. after death, and/or samples can be taken from a living subject.

As used herein, terms “treatment”, “treating”, “palliating”, “alleviating” or “ameliorating”, used interchangeably, refer to an approach for obtaining beneficial or desired results including, but not limited to, therapeutic benefit. By therapeutic benefit is meant eradication or amelioration or reduction (or delay) of progress of the underlying disease being treated. Also, a therapeutic benefit is achieved with the eradication or amelioration or reduction (or delay) of progress of one or more of the physiological symptoms associated with the underlying disease such that an improvement or slowing down or reduction of decline is observed in the patient, notwithstanding that the patient can still be afflicted with the underlying disease.

As used herein, the term “implantable cell delivery device” is interchangeably used with “implant device”, “cell delivery device”, “implantable device”, “macro-encapsulating implant” or simply “device” or “implant” and refers to an enclosure suitable for retaining cells and which enclosure is intended for implanting in a subject. The device thus serves as a vehicle to transplant cells in a subject. Therefore it may be assumed that the device is of a material suitable for implanting in a subject and that the device is constructed such that it is suitable to contain living cells.

As used herein the term “open” when referring to the implantable cell delivery device implies that the device has one or more openings that allow vascularization or vascular ingrowth in the device. For the purpose of the invention, the openings refer to the pores. The term “open” does thus refer to the pores that are present to allow nutrient diffusion. As such the pore size of an open device is such that it allows for vascular ingrowth in the device, and further allows cells to enter the device. Thus an open type device has pores with a pore size sufficiently large to allow vascular ingrowth and cell to enter the device. Similarly the term “closed” when referring to the implantable cell delivery device implies that the device allows the diffusion of nutrients and oxygen, but does not allow for vascularization inside the device or for cells to enter the device, and thus only comprises pores or openings too small to allow vascularization.

As used herein the terms “vascular ingrowth” and “vascularization” are used interchangeably and refer to angiogenesis of vasculature through an opening of the device, such that the newly developed blood vessel at least partly enters the inner space of the device, allowing the exchange of nutrients and oxygen, among others.

When used herein, the term islet cells refers to pancreatic islet cells, also known as islets of Langerhans, and comprising among other beta cells producing insulin. The terms also includes primary islets.

When used herein, the term “cells” when referring to cells intended to be used in the implant device refers to cell clusters or organoids. Further when referring to “cell clusters”, the term is understood to comprise “organoids”. Thus the term “cluster” when referring to cells is regarded as a genus for the species “organoid”. Thus where referred to cell clusters herein, also organoids are included.

When used herein, the term “biocompatible” refers to the ability of a biomaterial to perform its desired function with respect to a medical therapy, without eliciting any undesirable local or systemic effects in the recipient or beneficiary of that therapy. Non limiting examples of undesirable local or systemic effects are toxic or injurious effects on biological systems.

When used herein, the term “biomaterial” refers to a substance that has been engineered to interact with biological systems for a medical purpose. Specifically, when used herein biomaterial refers to the implantable cell delivery device or its individual components.

### DETAILED DESCRIPTION OF THE INVENTION

The section headings as used herein are for organizational purposes only and are not to be construed as limiting the subject matter described.

A portion of this invention contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent invention, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention relates, unless otherwise indicated. Other specifically defined terms are to be construed in a manner consistent with the definition as provided herein. The preferred materials and methods are described herein, although any methods and materials similar or equivalent to those described herein can be used in the practice for testing of the present invention.

Unless otherwise defined, all technical and scientific terms used herein have the same meaning as commonly understood by a person of ordinary skill in the art.

Present invention relates to open type implantable cell delivery devices intended to transplant cells in a subject, for example as a means of treating a disease. Exemplary cells that could be used in transplantation therapy are pancreatic islet cells in the treatment of diabetes, but other cells are known to the skilled artisan which could be used in therapeutic methods by transplantation.

Implantation of any polymer device will lead to the formation of a fibrous layer, as a final attempt of the human body to isolate and prevent outspread of the foreign body. Minimizing the fibrous tissue layer around a cell delivery device is of key importance, as it will increase the diffusion distance of oxygen and nutrients towards the implant and impairs the ingrowth of vasculature towards the cells, both of which will result in diminished cell functioning. A biocompatible biomaterial screening for pancreatic beta cells indicated that polyvinylidene fluoride (PVDF) is one promising candidate. PVDF is currently used in the clinic as suture material, a small cornea aperture inlay and as a mesh for hernia repair. PVDF is highly biocompatible and is associated with lower fibrous tissue formation compared to conventional polymers such as polypropylene. Other biocompatible biomaterials are known to the person skilled in the field, such as but not limited to polycarbonate (PC), polypropylene (PP), poly(ethylene terephthalate (PET), poly(vinyl chloride) (PVC), polyamide (PA), polyethylene (PE), polyimide (PI), polyacrylate, polyolefins, polysulfone (PSF), tetrafluoroethylene/polytetrafluoroethylene (PTFE), ePTFE (expanded polytetrafluoroethylene), polyethersulfone (PES), polycaprolacton (PCL), poly(methyl methacrylate) (PMMA), poly(lactic acid) (PLA) or combinations thereof.

Here the inventors describe the fabrication of an open microwell-array implantable cell delivery device from clinically approved PVDF that can be upscaled to clinically relevant sized implants. As proof of concept, mouse-sized implants were fabricated, seeded with primary rat islets and evaluated for islet viability and beta cell functionality during 7 days in vitro culture. The design of the mouse-sized devices was upscaled to rat-sized open islet implants, which were then fabricated and evaluated for rat islet or human islet viability and functionality during 7 days in vitro culture.

Therefore, in a first aspect the invention relates to an open type implantable cell delivery device for transplanting cells in a subject, comprising: —a bottom film having a surface area with a plurality of pores; —a top film having a surface area with a plurality of pores, positioned on top of the bottom film such that the top film substantially covers the bottom film to create an inner space; wherein the bottom film and the top film are formed of a biocompatible biomaterial, and wherein the bottom film comprises a plurality of microwells positioned to face the surface area of the top film with the open sides of said microwells. Preferably wherein the pore size of the bottom film and optionally the top film is such that it allows vascularization or vascular ingrowth in the device through the pores, and allow cells to enter the device.

In an embodiment the open type implantable cell delivery device further comprises a supporting structure positioned substantially around the surface area of the bottom film and the surface area of the top film such that the supporting structure is positioned in the plane of the surface areas of the top and the bottom films, wherein the bottom and the top film are attached to the support structure in one or more places such as to leave one or more openings between the top film, the bottom film and the support structures allowing contact between the inner space and the surroundings. Preferably the supporting structure is also formed from a biocompatible biomaterial.

In an embodiment the biocompatible biomaterial is selected from polyvinylidene fluoride (PVDF), polycarbonate (PC), polypropylene (PP), poly(ethylene terephthalate (PET), poly(vinyl chloride) (PVC), polyamide (PA), polyethylene (PE), polyimide (PI), polyacrylate, polyolefins, polysulfone (PSF), tetrafluoroethylene/polytetrafluoroethylene (PTFE), ePTFE (expanded polytetrafluoroethylene), polyethersulfone (PES), polycaprolacton (PCL), poly(methyl methacrylate) (PMMA), poly(lactic acid) (PLA) or combinations thereof.

As is of concern with many transplantation sites or implantations, proper (re-) vascularization is essential to ensure optimal survival and functioning of the graft. For example, pancreatic islets show a high metabolic activity and therefore require swift access to oxygen and nutrients to survive. It is therefore vital that the delivery devices are as thin and porous as possible to reduce both the diffusion distance and vasculature ingrowth distance into the implant. The aim of the present invention is therefore, among others, to fabricate an open macro-encapsulating cell delivery device to realize cell delivery, and to upscale the implant design towards clinically relevant device dimensions.

The unique microwell features of the implantable cell delivery device described herein allows control over the spatial distribution of cell clusters such as islets within the device, thereby effectively preventing further aggregation of multiple cell clusters into large cell aggregates and the formation of hypoxic cores in aggregated cell clusters. Herein, the ideal cluster to cluster (e.g. islet-islet) distance, the degree of overfilling of microwells and the possibility to stack multiple microwell layers on top of each other were evaluated to increase the islet packing density within the device. The strategies were investigated through a combination of in vitro experiments and in silico modelling of local oxygen levels surrounding islets. Predicted device dimensions of upscaled versions of the open device showed to be capable of housing clinically relevant islet numbers with device dimensions suitable for transplantation at the pre-peritoneal site. Although the models were based on islet based parameters, the principle can be applied to any other cell or organoid type to predict the optimal device dimensions.

The implantable cell delivery device according to the invention is intended to transplant cells in a subject. The examples provide data for a device for implanting islet cells, but it is understood that other cell types, mixtures of cell types, organoids or (parts of) tissue or organs can be included in the implantable cell delivery device. As the device is intended for implanting in a subject, there are certain limitations to e.g. the materials used which must be biocompatible as well as the dimensions of the device. It is understood that the dimensions may depend on the subject in which the device is intended to be implanted. When used herein, the term subject may refer to an animal such as a rodent or a mammal or a human. Therefore, the size limitations for an implantable cell delivery device are different for e.g. a mouse when compared to a human, however even within the same species differences in e.g. body size may influence the size of the implantable cell delivery device. The size of the device is further influenced by the cell type intended to be included in the implantable cell delivery device. The skilled person is capable to estimate an approximate desired size of the implant based on, among others, the subject and the cell type to be transplanted.

The implantable cell delivery device according to the invention comprises a bottom film having a surface area with a plurality of pores and a top film having a surface area with a plurality of pores, positioned on top of the bottom film such that the top film substantially covers the bottom film to create an inner space. The bottom film comprises a plurality of microwells, the opening of these wells facing the inner space. The wells are intended to hold the cells. Therefore, the inner space is preferably such that the cells (e.g. cell clusters or organoids) are more or less held in place in the wells by the top film so that the cells do not freely move inside the device. Both the top film and the bottom film have a plurality of holes (pores) allowing the diffusion of nutrients and oxygen towards the cells, and optionally, secreted factors from the cells out of the device. A non-limiting example of a secreted factor is insulin. Further the pore size is sufficiently large such that it allows vascular ingrowth and cells to enter the device.

When used herein, a film refers to a thin and flat material. When used herein, the surface area of the film refers to its face side. When used herein a pore refers to an opening or cavity in the film that completely penetrates the film and thus allows for the passage of e.g. molecules from one side of the film to the other. When used herein a pore is sufficiently large to allow vascular ingrowth and cells to enter the device.

The implantable cell delivery device according to the invention further optionally comprises a supporting structure positioned substantially around the surface area of the bottom film and the surface area of the top film such that the supporting structure is positioned in the plane of the surface areas of the top and the bottom films. The supporting structure may for example by oval or round, but it is understood that it may have any kind of shape. Ideally, the shape of the supporting structure follows the contours of the top and bottom films. For example, when the bottom and top film have an oval shape, the supporting structure is preferably an oval shaped ring following the edges of the bottom and top films. It is understood that the supporting structure may have openings, for example the supporting structure may also be U-shaped.

It is understood that the device may comprise additional supporting structures. For example, the open type implantable cell delivery device according to the invention may comprise one or more additional support structures, preferably wherein said one or more additional support structured are positioned more centrally with respect to the bottom and top films.

The function of the supporting structure is to provide some rigidity to the device. Although some degree of flexibility is desirable in an implantable cell delivery device, the structural integrity must be ensured. Because the bottom and top films must allow the diffusion of nutrients and oxygen, vascular ingrowth, there are limitations to the thickness of the films which in general are very thin and thus fragile. The supporting structure(s) help(s) to avoid tearing or rupturing of the films. Further, inclusion of the supporting structure prevents folding or bending of the device, which could otherwise lead to an increased inner space, which may cause cells to migrate out of their wells. Therefore, the supporting structure has a thickness which is generally substantially more than the thickness of the bottom and top films. For example, the thickness of the top and bottom films may each individually be between 5 and 50 μm thick, preferably between 10 and 30 μm more preferably around 15 μm, while the supporting structure may be around 75 to 500 μm thick, preferably between 100 to 400 μm more preferably around 200 μm thick.

The supporting structure further can serve as a scaffold for attaching the bottom and top films. The films may be attached to the supporting structure such that the supporting structure is sandwiched between the edges of the films, alternatively the films may be attached together on one side of the supporting structure, e.g. the top or the bottom side. The films may be attached for example by ultrasonic welding. It is understood that if no supporting structure is used in the device that the bottom and top films can be attached to each other directly.

The implantable cell delivery device according to the invention further has the bottom and the top film attached to the support structure in one or more places. The bottom and the top film may be attached to the support structure such as to leave one or more openings between the top film and/or bottom film and the support structure. The purpose of these openings is to allow one or more spaces or openings where vascularization of the implantable cell delivery device can occur. An advantage of the open type implantable cell delivery device is the option to allow vascularization in the device, resulting in better exchange of nutrients and oxygen, and uptake of factors secreted by the cells in the implantable cell delivery device (e.g. insulin). Therefore, the inner space of the implantable cell delivery device is in open connection with the outside through at least the plurality of pores and the one or more openings between the films and the supporting structure. Additionally or alternatively, the bottom and top films may be completely sealed to the support structure (meaning leaving no openings) but the pore size is selected such that the pores allow for vascularization (and cells to enter the device). Alternatively, both the pore size is sufficiently large to allow for vascular ingrowth and openings are provided between the bottom and top films and the support structure.

It is further envisioned that the top film and the bottom film are the same film which is folded upon itself. When used herein, the bottom film is defined as the film comprising the microwells, consequently the covering film is considered the top film, regardless of their actual position (e.g. top or bottom). When both film comprise microwells, either one of the films can be considered the bottom film.

It was found that the device is preferably constructed from a biocompatible biomaterial. A particularly preferred material is PVDF, as it has improved porosity compared to other suitable materials while maintaining mechanical strength. A further advantage is that PVDF is biocompatible, and thus does not trigger an immune response nor affect the cells in the device in a negative way. It is understood that the PVDF may be mixed with a suitable material, a non-limiting example being PVP. The device may however also be constructed from other biocompatible biomaterials, as different materials may have advantages depending on the specific use (e.g. location of implantation, size of the device, type of cells in the device, etc.). Other non-limiting examples of biocompatible biomaterials are polycarbonate (PC), polypropylene (PP), poly(ethylene terephthalate (PET), poly(vinyl chloride) (PVC), polyamide (PA), polyethylene (PE), polyimide (PI), polyacrylate, polyolefins, polysulfone (PSF), tetrafluoroethylene/polytetrafluoroethylene (PTFE), ePTFE (expanded polytetrafluoroethylene), polyethersulfone (PES), polycaprolacton (PCL), poly(methyl methacrylate) (PMMA), poly(lactic acid) (PLA) or combinations thereof.

The microwells are intended to hold either cell clusters and/or organoids. Therefore, in an embodiment the microwells have a diameter of 200-1000 μm, preferably of 250-950 μm, more preferably of 300-900 μm. Ideally the wells prevent aggregation of multiple cell clusters to such an extent that the centrally located cells in the aggregate start to necrotize from lack of nutrients or oxygen. The skilled person will appreciate that depending on the cell type, cell size, cell cluster or organoid the well size needs to be varied. It is understood that the terms “cell clusters and/or organoids” may refer to cultured cells or cells resected from a tissue or organ of a donor organism.

In a particularly attractive embodiment, the well size is approximately 300 to 500 μm in diameter, preferably 350 to 450 μm more preferably approximately 400 μm, as it allows cell clusters to be isolated in the wells. In alternative attractive embodiment, the wells have a diameter of between 600 and 1000 μm, preferably between 700 and 900 μm, more preferably approximately 800 μm in diameter, as it allows to include cells encapsulated in a hydrogel (also known as hydrogel capsules). An advantage of using hydrogel encapsulated cells is that it fixes the cells in the wells, preventing them from falling out or migrating away from the wells. A further advantage is that it prevents the immune system from reaching the encapsulated cells. However, bulk encapsulation of cells within a hydrogel will most likely result in too long diffusion distance as vasculature cannot grow into the hydrogel. The usage of hydrogel capsules is therefore an attractive alternative, ensuring short diffusion distances while still preventing the immune system from reaching the encapsulated cells. However, the main disadvantage of hydrogel capsules is that they are difficult to locate and recover after surgery. Therefore, the advantages of hydrogel encapsulation (no access of immune system) can be combined with the advantages of the open device, namely a retrievable construct with increased diffusion of nutrients and oxygen due to the small hydrogel capsules encapsulating the cell clusters. It is assumed that for most applications an immune response of the subject to the cells in the device leads to targeted destruction of the cells by the immune system and is thus not desirable. If however interaction of the immune system with the cells in the device is desirable a hydrogel should not be used to embed the cells.

It is understood that when using hydrogel encapsulated cells are used, the pore size may be even larger as the hydrogel will prevent the cells from leaving the device. Therefore in an embodiment the cells are encapsulated in hydrogel, and the pore size of the bottom film and optionally the top film of the device is between 5 and 200 μm, for example between 25 and 200, 50 and 190, 75 and 180, 100 and 170 or 125 and 160 μm.

In an embodiment the wells are spaced such that that the cell clusters inside the wells are approximately 300 μm apart, for example 200-400 μm apart, preferably 250 to 350 μm apart. It is understood however that spacing of the cell clusters depends on their size, meaning that larger cell clusters require larger spacing, to prevent local oxygen depletion. For example it was found that for cell clusters with a diameter of 50 μm virtually no spacing is required, while for a cell cluster of 100 μm a distance of approximately 100 μm suffices, for a cell cluster of 150 μm a distance of approximately 300 μm suffices. Cell cluster equal or larger than 200 μm in diameter showed insulin depletion irrespective of their islet-islet distance.

The pores in the top and bottom film allow diffusion of nutrients and oxygen to the cells in the device, in addition to diffusion of nutrients and oxygen as the result of ingrowth of blood vessels. Further the pores allow for vascular ingrowth into the inner space of the device and for cells to enter the device. Ideally the pore size and pitch are chosen such as to allow maximal diffusion and vascular ingrowth while maintaining structural integrity and preventing the cell clusters from exiting the device. Therefore, in an embodiment the pore size of the bottom film and optionally the top film is between 5 and 100 μm, preferably between 10 and 80 μm more preferably between 15 and 60 μm most preferably between 20 and 55 μm. Optionally the pores have:
an average pitch between 10 and 1000 μm, such as for example 50 or 100 μm, with the proviso that the pitch is larger than the pore size; or
an average pore density of between 25 to 500 pores per mm², preferably between 40 to 250 pores per mm².

It is understood that the pore size is limited by the size of the cell clusters and/or organoids contained in the device, thus preferably the pore size does not exceed the size of the cell cluster or organoid intended to be contained in the device. Further, it is understood that if cell clusters are embedded in a hydrogel, the device may allow for a bigger pore size, even exceeding the size of the cell clusters. The pore size in the top and the bottom film may be the same or may be different. For example, if the device includes a mesh between the top and bottom film preventing the cells from leaving the wells, the top film can have a pore size which is larger than the pore size of the bottom film, and the pore size of the top film may exceed the cell (cell cluster or organoid) size. In the latter case the pore size of the top film is only constrained by the structural integrity of the film. When used herein the term pore size refers to its diameter.

It is understood that diffusion of nutrients and oxygen is dependent on vascular ingrowth into the device as well as determined by the total surface area of the pores, meaning a function of the pore size and number of pores in the top or bottom film. It is therefore understood that the pitch of the pores is, depending on the pore size, between 10 and 1000 μm, preferably between 20 and 900 μm, more preferably between 40 and 800 μm most preferably between 50 and 750 μm. It is further understood that the pitch should be larger than the pore size. When used herein, pitch is used to describe the distance between the centres of repeated elements, in this case pores in the film. It is assumed that the pores are more or less evenly distributed. If the pores are not evenly distributed the term pitch refers to the average distance between neighbouring pores.

Alternatively, the number of pores can be expressed as the number of pores per square mm (pore density). Depending on the pore size, the pore density is preferably between 25 to 600, more preferably 25 to 500, more preferably 40 to 550, more preferably between 40 and 400, more preferably 50 to 500, even more preferably between 50 and 300, even more preferably between 75 to 450 or 75 and 300 pores per mm².

In an embodiment the microwells comprise cell clusters and/or organoids, preferable wherein the cell clusters are endocrine cells or cytokine producing cells or clusters thereof, preferably wherein the cell clusters are selected from islet cells, kidney cells, thyroid cells, thymic cells, testicular cells, pancreatic cells, or preferably wherein the organoid is selected from an intestinal organoid, a gastric organoid, a thyroid organoid, a thymic organoid, a testicular organoid, a hepatic organoid, a pancreatic organoid, an epithelial organoid, a lung organoid, a kidney organoid, a gastruloid (embryonic organoid), a blastoid (blastocyst-like organoid), a cardiac organoid, a retinal organoid or a glioblastoma organoid. The cell clusters may also refer to a resected piece of tissue or organ, for example obtained from a donor organism. Alternatively the cell clusters or organoids may be obtained from a cell line or stem cells, such as but not limited to induced pluripotent stem cells.

It is envisioned that the device is particularly suited for implanting in a subject with cells that secrete a substance, such as a hormone or cytokine or any therapeutic protein. Therefore, the cell clusters and/or organoids preferably comprise or consist of endocrine or cytokine producing cells.

In an embodiment the cell clusters or organoids contained in the microwells have a diameter of 40 to 300 μm, preferably 50-250 μm. It is understood that ideally the microwells comprise one cell cluster or organoid each, therefore when substantially all microwells comprise at least single cell cluster or organoid, in order for efficient use of space in the device the cell cluster or organoid is preferably between 150 and 300 μm, preferably 200 and 250 μm in diameter. Alternatively the microwells can be filled with multiple cell clusters or organoids, however it is understood that to prevent local oxygen depletion then the cell cluster or organoid size preferably is kept smaller. For example when the microwell contains two cell clusters or organoids, the diameter is preferably between 40 and 150, more preferably between 50 and 100 μm in size. When containing three or even four cell clusters or organoids per microwell, the diameter is preferably between 40 and 120, preferably between 50 and 100 μm in size.

In an alternative preferred embodiment, the microwells have a diameter of 600-1000 μm, preferably 700-900 μm, more preferably 750-850 μm. Such large well diameters are useful for situations wherein the cell clusters and/or organoids are encapsulated by a hydrogel, or wherein the cell clusters or organoids are large in size and thus require large wells.

An additional advantage of using PVDF as the material to manufacture the device is that it allows spot welding. Therefore in an embodiment the top and bottom film are attached to the support structure by spot welding. Spot welding has the advantage that no additional materials need to be used such as a glue, which may trigger an immune system, may not be biocompatible or even toxic, or may dissolve over time resulting in structural failure of the device.

It is further envisioned that a marker for imaging is included in or on the device. This may be advantageous as it allows for locating the device in a subject without the need for surgical procedures. Therefore, in an embodiment the device comprises one or more markers for imaging, preferably wherein said one or more markers comprise a radiopacifier infused in or coated on the PVDF of the top film, the bottom film and/or the support structure, more preferably wherein said radiopacifier is barium based such as barium sulfate, bismuth based such as bismuth trioxide, bismuth subcarbonate or bismuth oxychloride, or wherein the radiopacifier is tungsten or graphene oxide.

When used herein, a radiopacifier, also referred to as radiocontrast material, is a substance that is opaque for the radio- and x-ray waves portion of the electromagnetic spectrum, meaning a relative inability of those kinds of electromagnetic radiation to pass through the particular material. Non-limiting examples of radiocontrast materials include titanium, tungsten, barium sulfate, bismuth oxide and zirconium oxide. Some solutions involve direct binding of heavy elements, for instance iodine, to polymeric chains.

It is further envisioned that it may be advantageous to include a drug with the device. Therefore, in an embodiment the device comprises a drug infused in or coated (e.g. by dipping the device in a solution of the drug) on the PVDF of the top film, the bottom film and/or the support structure. For example, the device may be coated with an immune suppressing drug to prevent degradation of the cells in the device by the immune system or a drug that reduces the fibrotic response. Alternatively, a therapeutic drug may be included as a co-treatment in case the device is implanted as a treatment option in the subject. Non-limiting examples are chemotherapeutical agents for treatment of cancer, immune checkpoint inhibitors, cell stress inhibitors aiding in cell cluster and/or organoid survival in the early post-surgery period, or imaging markers for tracking the implant post-surgery. Further envisioned is the inclusion of angiogenic factors to promote vascular ingrowth in the device.

It is understood that the size of device can be scaled depending on the intended application (e.g. treatment method or type of cells contained in the device) and based on the subject. It will be clear to the skilled person that a device intended for implantation in a human subject need to be larger than a device intended to be implanted in a rodent. Because the device is essentially two-dimensional, meaning existing of a single plane with wells, it is anticipated that for some applications the device needs to be scaled to an impractical size in larger mammals such as humans. Although in theory multiple smaller versions of the device can be used, in practice it is not desirable to implant multiple devices at the same or different locations. It is therefore further envisioned that multiple smaller versions of the device can be stacked together. Therefore, in an embodiment, the device comprises two or more stacked versions of the open type implantable cell delivery device as defined herein stacked on top of each other and separated by a spacer. The spacer essentially functions to create distance between the individual devices. Therefore in an embodiment the spacer allows for a spacing of 200-800 μm between the different stacked devices, preferably between 250 and 700 μm and more preferably between 250 and 650 μm. It was found that when using a stack of two devices a spacing of 250-350 μm suffices between the devices. When using more, for example three or more stacked devices it may however be beneficial to increase the spacing, therefore when the device comprises three or more stacked versions of the device the spacing is preferably between 250 and 750 μm, more preferably 300-700 μm, 400-650 μm or even 450-600 μm. In a more preferred embodiment, the device comprises two stacked versions of the open type implantable cell delivery device as defined herein, as it was found that using a stack of two allows for optimal oxygenation of the cells while increasing cells density in the device.

Two or more devices can in principle be stacked to obtain a more compact design, as long as proper diffusion of nutrients and oxygen to the cells is ensured. This becomes particularly relevant when three or more layers are stacked with respect to the middle layers. It is therefore found by the inventors that stacked layers should be separated by a spacer. Preferably the spacer is constructed such that the space between layers is not completely enclosed by the spacer. Therefore, either several small spacers may be used, or the spacer may have openings. The spacer may be regarded as an additional support structure, therefore when used herein the spacer may also be referred to as “additional support structure”. Preferably, the spacer is also constructed from PVDF to ensure biocompatibility. It is further envisioned that the support structures and the spacer(s) (additional support structure) are one continuous structure.

It is envisioned that the device may be used in a medical method or a method of treatment. Therefore, in a second aspect the invention relates to the open type implantable cell delivery device according to the invention for use in the treatment, prevention or amelioration of a disease. The device preferably comprises cells, more preferably cell clusters or organoids, therefore, in an embodiment the invention relates to the open type implantable cell delivery device comprising cells, preferably cell clusters and/or organoids, according to the invention for use in the treatment, prevention or amelioration of a disease. Alternatively, the invention relates to a method of treating, preventing or ameliorating a disease or a condition in a subject in need thereof, the method comprising implanting the device comprising cells, preferably cell clusters or organoids, in the subject.

Several treatment options are envisioned for the device. For example, the device may be used in the treatment of diabetes. Therefore, in an embodiment, the treatment is treatment of diabetes, preferably type 1 diabetes. Preferably when the treatment is treatment of diabetes the device comprises insulin secreting cells, such as islet cells or cells engineered to secrete insulin.

It will however be clear to the skilled person that use of the device is not limited to treatment of diabetes, as the device allows for incorporation of any type of cell, cell cluster or organoid. As the open type device allows for ingrowth of the vasculature it is particularly suitable for treatment options where administration of an exogeneous factor is desirable. Non-limiting examples of exogeneous factors include peptides and proteins such as insulin, glucagon, cytokines, growth factors, hormones, carbohydrates, and clotting factors.

Therefore the device may be used in the treatment of immune related disorders such as Multiple myeloma, Melanoma, Rheumatoid arthritis, Inflammatory bowel disease, Lupus, Scleroderma, hemolytic anemia, Vasculitis, Type 1 diabetes, Graves' disease, Multiple sclerosis, Goodpasture syndrome, Pernicious anemia, myopathy, Lyme disease, Severe combined immunodeficiency (SCID), DiGeorge syndrome, Hyperimmunoglobulin E syndrome (also known as Job's Syndrome), Common variable immunodeficiency (CVID), Chronic granulomatous disease (CGD), Wiskott-Aldrich syndrome (WAS), Autoimmune lymphoproliferative syndrome (ALPS), Hyper IgM syndrome, Leukocyte adhesion deficiency (LAD), NF-κB Essential Modifier (NEMO) Mutations, Selective immunoglobulin A deficiency, X-linked agammaglobulinemia (XLA; also known as Bruton type agammaglobulinemia), X-linked lymphoproliferative disease (XLP), Ataxia-telangiectasia or Acquired immunodeficiency syndrome (AIDS). Further, the device may be used in the treatment of a growth factor related disease such as cancer. Further, the device may be used in a hormone or endocrine related disorder such as Adrenal insufficiency, Addison's disease, Cushing's disease, Cushing's syndrome, Gigantism (acromegaly), Hyperthyroidism, Grave's disease, hypothyroidism, Hypopituitarism, Multiple endocrine neoplasia I and II (MEN I and MEN II), Polycystic ovary syndrome (PCOS) or Precocious puberty. Further, the device may be used for the treatment of a clotting disorder such as Factor V Leiden, Prothrombin gene mutation, Deficiencies of natural proteins that prevent clotting (such as antithrombin, protein C and protein S), Elevated levels of homocysteine, Elevated levels of fibrinogen or dysfunctional fibrinogen (dysfibrinogenemia), Elevated levels of factor VIII and other factors including factor IX and XI, Abnormal fibrinolytic system, including hypoplasminogenemia, dysplasminogenemia and elevation in levels of plasminogen activator inhibitor (PAI-1), Cancer, Obesity, Pregnancy, Supplemental estrogen use, including oral contraceptive pills (birth control pills), Hormone replacement therapy, Prolonged bed rest or immobility, Heart attack, congestive heart failure, stroke and other illnesses that lead to decreased activity, Heparin-induced thrombocytopenia, Antiphospholipid antibody syndrome, Previous history of deep vein thrombosis or pulmonary embolism, Myeloproliferative disorders such as polycythemia vera or essential thrombocytosis, Paroxysmal nocturnal hemoglobinuria, Inflammatory bowel syndrome, HIV/AIDS or Nephrotic syndrome.

In a third aspect the invention relates to a method of constructing open type implantable cell delivery device, the method comprising: providing a bottom film having a surface area with a plurality of pores and further comprising a plurality of microwells; position a top film having a surface area with a plurality of pores on the bottom film such that the openings of the microwells face the top film, to create an inner space between the bottom and top film in open contact with the microwells; positioning a support structure substantially around the assembly of bottom and top films in the same plane as the films such that the support structure at least partly overlaps with the edges of bottom and the top films; spot welding the bottom and the top films in two or more places to attach the bottom and top film to the support structure such as to leave several opening through which the inner space is accessible. The pore size of the bottom film and optionally the top film is such that it allows vascularization or vascular ingrowth in the device through the pores. The pore size further allows cells to enter the device.

When used herein, the term “spot welding” preferably refers to ultrasonic spot welding. Ultrasonic spot welding is an industrial process whereby high-frequency ultrasonic acoustic vibrations are locally applied to workpieces being held together under pressure to create a solid-state weld. It is commonly used for plastics.

In an embodiment, the bottom and top film are attached to the support structure with 2 or more spot welds, preferably at least 3, 4, 5, 6, 7, 8, 9, 10 or more such as 3 to 50, 4 to 40, 5 to 30, 6 to 25, 7 to 20 or 8 to 15. It is understood that the amount of welds are defined by the size of the device, and should be chosen such that the openings remain large enough for vascular ingrowth (due to spacing of the wells) but small enough to ensure structural integrity.

The microwells in the bottom layer may be formed by (micro-) thermoforming, as PVDF is particularly suited for thermoforming processes.

The inventors are not aware of any other open type implantable cell delivery device which comprises wells. A reason may be that there are several obstacles for the construction of such device such as that it is not straightforward how the cells can be loaded in the device. One method would be to seed the cells in the bottom layer prior to assembly of the device, however there are several impracticalities, as the device is preferably shipped in assembled form to the end user. Moreover, welding polymer layers near cells will lead to severe loss of cell viability. To overcome these issues, the inventors have developed a method of seeding the device which does not require assembly of the device after seeding, meaning the device is seeded when completely assembled. Therefore, in a fourth aspect the invention relates to a method of seeding an open type implantable cell delivery device as defined in in the first aspect or obtained or obtainable by the method according to the third aspect with cells, the method comprising: connecting a container for cells with a first end of a tube, and inserting the second end of the tube through an opening of the open type implantable cell delivery device into the inner space such that the inner space is in open connection with the container; clamping the exterior of the open type implantable cell delivery device such that all remaining openings are sealed; loading the container with cells or cell clusters suspended in a suitable medium; allowing the cells to flow from the container through the tube into the inner space of the open type implantable cell delivery device while excess medium is drained through the pores.

It was found that by clamping the openings between the top and bottom layer shut except for a single opening, a cell suspension can be drained by gravity flow in the device. To enable this the cell suspension is taken in a container and slowly drained through a tube, where the other end of the tube is inserted through the single opening in the interior of the device (between the top and bottom film). Because the liquid can drain through the pores in the device, the cell suspension can simply flow through gravity allowing the cells to be deposited in the microwells of the device while the liquid drains out. The method effectively prevents that the cells will be lost during seeding. It is understood that instead of by gravity a pump or syringe may also be used to insert the cells suspension in the device.

Therefore, in an embodiment the cells are allowed to flow through the tube into the inner space of the open type implantable cell delivery device by gravity.

It is understood that preferred numbers provided herein for microwell size, pore size, cell cluster or organoid diameter, spacer size or well distance are based on the data obtained with islet cell clusters. Although this data may be extrapolated to clusters or organoids of different cell types, it is possible that ideal values are different. The skilled person is aware that the methods described herein, particularly in Example 2 below, may be adapted for different cell types to obtain ideal parameters for microwell size, pore size, cell cluster or organoid diameter, spacer size or well distance for the particular cell or organoid type.

### BRIEF DESCRIPTION OF THE FIGURES

FIG. 1: Step-by-step microfabrication processing steps of polymer films to create porous, thermoformed microwell-array bottom films for islet encapsulation. Scanning electron microscopy (SEM) images of (top row) solvent-casted PVDF followed by (bottom row) laser-drilling of PVDF films and thermoforming (E, F). Views are glass side (A), air side (B), top (D, E) and cross sections (C, F). (G, H) Quantification of average pore size in both PVDF and PolyActive before (G) and after (H) micro-thermoforming, as well as the well depth of micro-thermoformed films (1). Data are represented as mean±SD, * p<0.05

FIG. 2: Assembly of an open PVDF microwell-array implant. A) Cross section of the implant design (center) with details depicted in scanning electron microscopy (SEM) micrographs of the porous lid (top left, top view), microwell-array thermoformed films (top right, cross section), surface of support ring (bottom left, top view) and ultrasonically welded point seal (bottom right, top view). B) Mouse-sized implant (sealed at 4 points) with 300 microwells contained in an area with a diameter of 8 mm. The insert illustrates the distribution of rat islets seeded within this mouse-sized implant. (C) Failure stress (D) Peak stress (E) Failure strain and (F) Young's modulus of polymer thin films (N=10) and ultrasonic (US) welded seal between a film and support ring (N=6). Data are represented as mean±SD.

FIG. 3: Rat islets remain viable and functional over 7 days of culture in mouse-sized implants. (A-1) Live/dead stainings of islets cultured as free-floating controls at day 1 (top row) and day 7 (middle row) and islets cultured in the implant (bottom row) at day 7. Fluorescence microscopy images (two left columns) show live (green; A, D, G) and dead (red; B, E, H) islets. Brightfield microscopy (C, F, I) shows the islets in their culture environment. (J) Quantification of live/dead staining (A-1) of islets show similar viability for those seeded in the implant compared to free-floating controls at day 7. (K) Secreted insulin of rat islets during a glucose-stimulated insulin secretion (GSIS) test in which islets were cultured alternatively in 1.67 mM, 16.7 mM and 1.67 mM glucose. (L) Stimulation index of rat islets over time. Islets displaying a stimulation index >2 (red line) are considered functional. Data (>10 islets for viability, n=3 for insulin secretion) are represented as mean±SD, * p<0.05.

FIG. 4: Upscaling of the open type implantable cell delivery devices towards large animal- and human-sized implants. Implant dimensions were enlarged towards rat- (holding 3000 IEQ), mini-pig- (holding 13,000 IEQ) and human-sized implants (holding 200,000, 450,000, or 700,000 IEQ). Implant dimensions are given as minor diameter×major diameter. Each square in the underlying grid pattern represents 1 cm². Several implant dimensions are shown in which islets are distributed through one implant with 1 IEQ/well (black), one implant with 2 IEQ/well (grey) or two implants with 2 IEQ/well (white).

FIG. 5: Rat islets remain viable and functional over 7 days of culture in rat-sized implants. (A-F) Live/dead stainings at day 7 of culture of controls (free floating islets, top row) and islets cultured at density of 500 IEQ/cm² (300 IEQ/mL) in the implant (second row). Fluorescence microscopy images (two left columns) show live (green; A, D) and dead (red; B, E) islets. Brightfield microscopy (C, F) shows the islets in their culture environment. G) Quantification of live/dead staining (A-F) of islets show similar viability for those seeded in the implant compared to free-floating controls at day 7. H) Secreted insulin of rat islets during a glucose-stimulated insulin secretion (GSIS) test in which islets were cultured alternatively in 1.67 mM, 16.7 mM and 1.67 mM glucose. I) Stimulation index of rat islets over time. Islets displaying a stimulation index >2 (red line) are considered functional. Data (>10 islets for viability, n=3 for insulin secretion) are represented as mean±SD, * p<0.05.

FIG. 6: Human islets remain viable and functional over 7 days of culture in rat-sized implants. (A-I) Live/dead stainings at day 7 of culture of controls (free floating islets) at 150 IEQ/cm² (top row), controls at 600 IEQ/cm² (second row) and islets cultured at density of 600 IEQ/cm² in the implant (third row). Fluorescence microscopy images (two left columns) show live (green; A, D, G) and dead (red; B, E, H) islet cells. Brightfield microscopy (C, F, I) shows the islets in their culture environment. J) Quantification of live/dead staining (A-I) of islets show similar viability for those seeded in the implant compared to free-floating controls at day 1 and 7. K) Secreted insulin of human islets during a glucose-stimulated insulin secretion (GSIS) test in which islets were cultured alternatively in 1.67 mM, 16.7 mM and 1.67 mM glucose. L) Stimulation index of rat islets over time. Islets displaying a stimulation index >2 (red line) are considered functional. Data (>10 islets for viability, n=3 samples for insulin secretion) are represented as mean±SD, * p<0.05.

FIG. 7: Process for implant assembly through ultrasonic welding. A) Branson LPX manual ultrasonic welding system. B) Ultrasonic (US) welding procedure depicting assembly of the open implant (1), with a support ring topped with a thermoformed PVDF film and porous PVDF lid. The sonotrode at the tip of the manual welder causes high-frequency ultrasonic acoustic vibrations at 40 kHz that are transduced to the polymer films, inducing local melting (2) and therefore annealing of the PVDF films (3). (C, D) US welding guides for mouse-sized (C) and rat-sized (D) implants consisting of a stainless-steel bottom and Teflon top. The red cylinder represents the tip of the US welder. E, F) Actual US-welded implants produced using these molds: E) a 4-point sealed mouse-sized implant and F) a 7-point sealed rat-sized implant.

FIG. 8: Seeding procedure for open type cell delivery devices. (A) Cell seeding set-up for gravity-based cell seeding, including a retort stand and burette clamp, cell container (syringe), stop cock, feeding catheter and cell seeding clamp. (B) Top view of the device with (Top) The feeding catheter inserted through the seeding inlet, (Middle) Cell seeding without clamping the exterior border leads to cell loss, as fluid will follow the path of least resistance at the large openings in between the point seals, (Bottom) Cell seeding with a seeding clamp, preventing the loss of cells at the exterior of the device. (C) Components of seeding tool, including screws and wingnuts to tighten the clamps. Clamps hold cutouts for silicon rings, which ensures tight but mild clamping of the open type cell delivery device. A nut is placed on the screws close to the seeding inlet, to prevent loss of fluid through the seeding inlet by creating a tilt. Examples of seeding clamps for (D) mouse-sized or (E) rat-sized open type cell delivery devices.

FIG. 9: Laser-micromaching of PVDF does not burn the chemical composition of PVDF films. (A) Backscatter image of locations at which EDX was performed. Carbon and Fluor content of PVDF films and depicted either as atomic % (B) or weight % (C). The absence in rise of carbon content indicates that the materials are not burned.

FIG. 10: Step-by-step alterations of PolyActive leads to microwell dimensions similar to PVDF films. Scanning Electron Microscopy (SEM) micrographs of (top row) solvent casted PolyActive followed by (bottom row) laser-drilling of PolyActive films and thermoforming (E, F). Views are glass side (A), air side (B), top (E) and cross sections (C, F). Laser-drilled PolyActive films show a pore diameter of 15 μm (D, stereomicroscopy image). Mechanical properties of PVDF and PolyActive thin films: (G) Young's modulus, (H) Peak stress, (I) Failure stress, and (J) Failure strain.

FIG. 11: Optimization of a macro-encapsulating, open islet delivery strategy for improvement of clinical islet transplantation. A) Working principle of an extrahepatic microwell-array islet delivery device. Pancreatic islets are distributed over microwells, preventing aggregation of islets to form larger constructs with necrotic cores. The porous nature of the device allows swift revascularization of pancreatic islets, maintaining islet viability and functionality. A rise in blood sugar levels can therefore be compensated by the release of insulin by transplanted pancreatic islets. B) The current planar configuration of the device leads to considerable device dimensions that are surgically challenging to implant. C) Potential upscaling possibilities with an increased islet packing density for larger devices are stacking of layers, tight packing of microwells and overfilling of microwells. D) The domain and oxygen supply boundary conditions of the computational model used to simulate local oxygen levels surrounding islets. E) Oxygen values were simulated over a mesh allowing simulation of oxygen gradients throughout the microwells. The mesh was more refined at the islet interface.

FIG. 12: Hypoxia staining intensity increases with increasing INS1E pseudoislet diameter. Seeding and aggregation of single INS1E cells over an incubation period of 3 days in 200 um diameter agarose chips A) 50 cells, B) 100 cells, C) 250 cells, or 400 um diameter agarose chips D) 500 cells, E) 750 cells, F) 1000 cells. Scale bar represents 400 μm. G) Quantification of pseudoislet diameter. Pseudoislets were stained for hypoxia (green) and Hoechst (blue) after either hypoxia (5% O₂, H) or normoxia (21% O₂, J) culture. Staining intensity was then quantified, and expressed as signal to noise ratio (I for hypoxia, K for normoxia). The red bar illustrates the hypoxia threshold. Data are represented as mean±SD, * p<0.05.

FIG. 13: The importance of islet diameter on local oxygen levels as determined through both in vitro hypoxia staining of human islets and an in silico computational O₂ consumption model. Human islets were stained for hypoxia (green) and Hoechst (blue) after either hypoxia (5% O₂, A) or normoxia (21% O₂, B) culture. Scale bar represents 150 μm. C) Hypoxia staining intensity was quantified and expressed as signal to noise ratio. Data are represented as mean±SD, * p<0.05. Computational oxygen consumption modeling results for islets with diameter of D) 50 μm, E) 100 μm, F) 150 μm, G) 200 μm, H) 250 μm. 1) Oxygen levels were visualized over a line drawn through the center of the two islets displayed in Figures D-H).

FIG. 14: The optimal distance between two islets depends on their diameters. Local oxygen levels surrounding differently sized islets (50-250 μm in diameter, Y-axis) distanced between 0-500 μm (X-axis) from each other. Hypoxia threshold was 5% O₂ (light blue).

FIG. 15: Microwells can be overfilled with small pseudoislets without causing severe O₂ competition. A) Simulation of differently sized islets that were held within an area similar to a microwell (400 μm wide and 250 μm high). Islet diameter ranged between 50 μm (left column), 100 μm (middle column) and 150 μm (right column). Islet density ranged between two islets (first row), three islets (second row) or four islets (third row) per microwell. Some representative images of INS1E pseudoislets cultured under normoxic conditions aggregated together with islet diameters around B) 50 μm, C) 100 μm and D) above 150 μm. Hypoxia was only observed in the largest islet diameter group, given that the hypoxia threshold for INS1E cells (SNR=3.0) was crossed.

FIG. 16: Stacking of multiple device layers lead to hypoxia in three-layered devices. Illustrations of a specific device assembly (left), followed by the simulation of local O₂ levels of 150 μm diameter islets (middle), and quantification of local oxygen levels over the dashed vertical line drawn through the islet(s) in the middle of the construct (right). First row: one-layered device, second row: double-layered device with 300 μm distance between layers, third row: double-layered device with 600 μm between layers, fourth row: three-layered device with 300 μm distance layers, fifth row: three-layered device with 600 μm between layers.

FIG. 17: The optimal packing density for microwell-array islet delivery devices. A) The optimal design and seeding distribution of islets in a double-layered microwell-array islet delivery device. B) Top view of a sentinel double-layered microwell device consisting with a 300 μm distance between layers, scale bar represents 1 cm (left). A frontal view of the device inserted with two feeding catheters illustrating the loading possibilities for each individual layer of the double-layered construct (middle). Layers were ultrasonically connected through point welding, leading to open edges of the device that allow free oxygen diffusion and tissue ingrowth (right).

### EXAMPLES

### Example 1

### Materials and Methods

### Polymer Film Fabrication

Thin films of PVDF (medical grade Kynar 720, Solvay) were solvent casted, as described in Li, M., et al., Controlling the microstructure of poly(vinylidene-fluoride) (PVDF) thin films for microelectronics. Journal of Materials Chemistry C, 2013. 1(46): p. 7695-7702. A 15% (w/w) polymer solution was prepared in dimethyl formamide (DMF, Sigma-Aldrich). An automatic film caster (Elcometer K4340M12) located within a humidified-controlled box was equipped with a glass plate, and preconditioned at a temperature of 100° C. and 10% humidity. The PVDF-DMF solution was then casted on the glass plate. A universal applicator (Elcometer K0003530M005) with a gap distance of 250 μm was run over the polymer solution at a constant speed of 5 mm/s to spread the polymer solution over the surface of the glass plate. The polymer film was then allowed to dry overnight under nitrogen gas flow, resulting in a 15 μm-thick film. Polymer films were incubated in demineralized water overnight to remove solvent residue and air-dried. PVDF films were made porous by laser micromachining with a UV-short pulse laser at a frequency of 25 kHz. Polymer films used for microwell bottom films were patterned with pores having a pore size of 25 μm and 50 μm pitch, while polymer films used as lids were patterned with a pore size of 40 μm and 100 μm pitch. Thin films of were also made from PolyActive, produced by Polyvation BV. The exact composition was 4000PEOT30PBT70, composed of poly(ethylene oxide) with a molecular weight of 4000, and weight percentage (wt %) of 30 wt % poly(ethylene oxide terephthalate) (PEOT) and 70 wt % poly(butylene terephtlalate) (PBT). PolyActive was dissolved in in a 65:35 (w/w) mixture of chloroform and 1,1,1,3,3,3-hexafluoro-2-isopropanol at a concentration of 15 wt % and casted on the film caster similarly to the PVDF, with the exceptions of using room temperature during casting and solvent leaching in ethanol. Polymer films were patterned with pores having a pore size of 15 μm and 50 μm pitch.

### Fabrication of Microwell Films

Thin films holding microwells were fabricated by means of micro-thermoforming. PVDF films were pressed in between a metal mold (Veld laser Innovations BV) and a 560 μm-thick polyethylene film functioning as backing material. The construct was placed in a hydraulic press (Atlas manual hydraulic press, Specac) and incubated for 2 min at 85° C. The pressure was subsequently increased to 30 or 35 kN for the mouse-, or rat-sized implants respectively. After a 10 min incubation, samples were removed from the hydraulic press and submerged in ethanol for 5 min to ease demolding.

A similar construct was made for PolyActive films, placed in the hydraulic press and incubated for 5 min at 85° C. The pressure was subsequently increased to 25 or 30 kN for the mouse-, or rat-sized implants respectively and samples were allowed to cool down in the press to 37° C. Samples were removed from the hydraulic press and submerged in ethanol for 5 min to ease demolding.

### Support Rings

A total of 2 g of PVDF pellets were loaded in a stainless-steel mold (200 μm-thick, 10×10 cm plate with negative 09 cm disc) and loaded in the hydraulic press. Samples were preheated for 1 min at 180° C. The pressure was increased and maintained at 20 kN for 1 min. Samples were then removed from the hydraulic press, allowed to cool for 5 min at room temperature and subsequently demolded, leading to 09 cm disks with a thickness of 200 μm. Next, the support rings were cut to the desired shape with a cutting plotter (Silhouette Cameo 4).

### Assembly by Ultrasonic (US) Welding

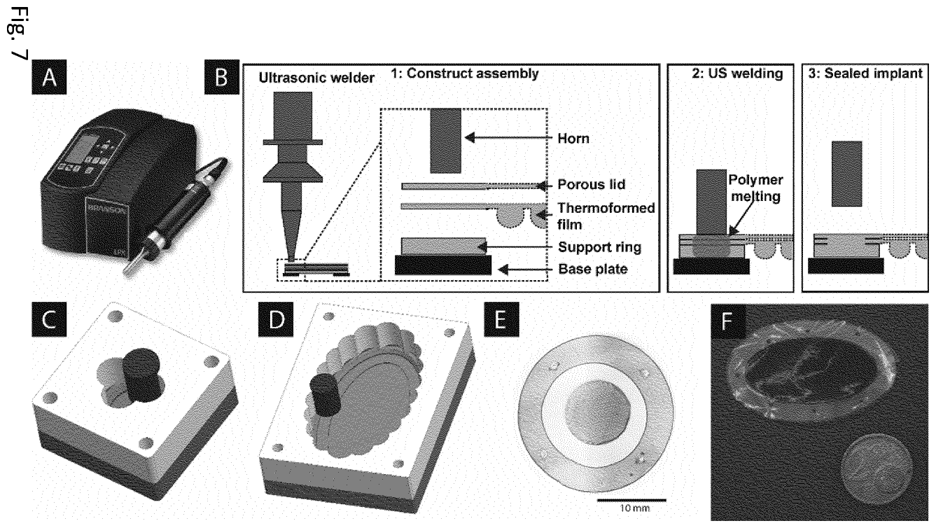

A custom-made US welding guide was used to control the assembly of open implants (FIG. 7). Firstly, a support ring was placed in the stainless-steel holder and covered with a porous micro-thermoformed bottom film and porous lid. A cloud-like pattern was milled in a Teflon cover plate and placed on the US welding guide, leading to either a 4-point or a 7-point seal, for the mouse- and rat-sized implants, respectively. PVDF layers were annealed by a 40 kHz manual Branson LPX US welding station at 75% amplitude for 1 s.

### Rat Islet Isolation

Animal experiments were approved by the institutional ethical committee on animal care and experimentation at Maastricht University and the Dutch central commission on animal work under application number AVD1070020186965. Rat islets were isolated from 10-week-old male Lewis rats. Rat pancreata were perfused with 0.25 mg/mL liberase (Roche) and kept on ice until digestion at 37° C. for 16 min. The digestion was stopped with quench solution (Hanks' balanced salt solution (HBSS) supplemented with 10 mM 4-(2-hydroxyethyl)-1-piperazineethanesulfonic acid (HEPES), 1% penicillin/streptomycin (P/S, 1000 U/mL), 2.5 mM CaCl₂·2H₂O, 4.2 mM NaHCO₃, 1 mM MgCl₂·6H₂O and 10% fetal bovine serum (FBS). Tissue was homogenized, filtered and washed with quench solution. Islets were purified with a ficoll gradient (Ficoll-Paque Plus, GE Healthcare) and centrifuged at 10° C. without brakes. Islets were washed with quench and medium (RPMI 1640 medium (11 mM glucose) supplemented with 10% FBS, 1% P/S, 10 mM HEPES and 1 mM sodium pyruvate). Islets were handpicked immediately after isolation and the following day. The purity and amount of islets were determined 24 h after isolation with dithizone staining (Sigma-Aldrich). The amount of islets was reported in islet equivalents (IEQ, the islet volume relative to islets with diameter of 150 μm) based on the conventional Ricordi method.

### Human Islets

A total of 20500 human islets of Langerhans with a purity of 80% were obtained from the Human Islet Isolation Laboratory at Leiden University Medical Center (LUMC, Leiden, the Netherlands) which has permission from the Dutch government to isolate human islets with clinical intend. Human islets that were not deemed suitable for clinical islet transplantation were used in these experiments, in accordance with Dutch law.

### Islet Seeding

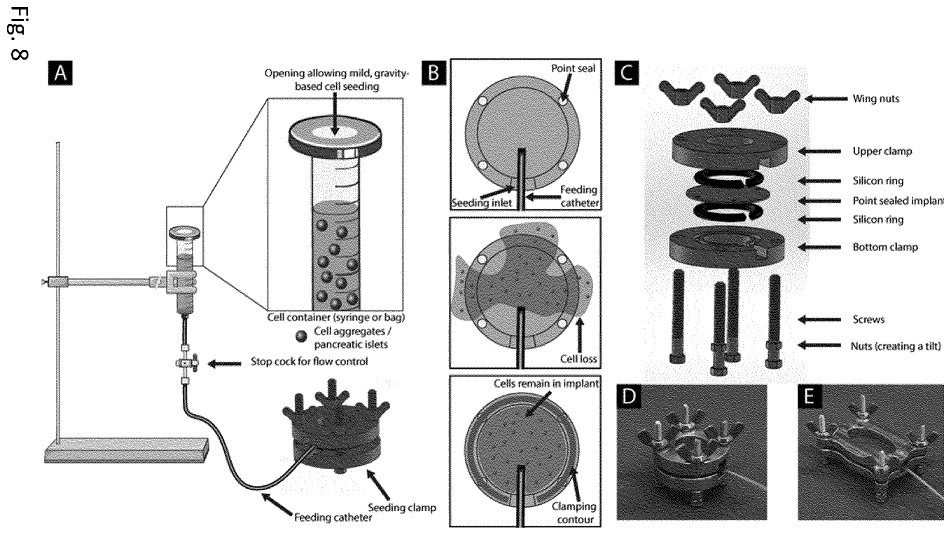

Free-floating control islets (rat islets 150 IEQ/cm² or 500 IEQ/cm², human islets 150 IEQ/cm² or 600 IEQ/cm²) were seeded inside a 12 mm Millicell cell culture insert (MERCK, 12 μm pore size) in a 24-well plate in 500 uL medium. The space between the point seals used to assemble the implant was intended to ease blood vessel ingrowth during future in vivo studies, but may make the implant prone to loss of islets during cell seeding. A seeding tool was designed to prevent islet loss by tightly clamping the outer border of the implant, ensuring that islet-loaded medium can only drain away through the pores in the microwell structures (FIG. 8). A Luer lock syringe was loaded with islets (200 IEQ/mL), connected to a 3.5 Fr blunt-tip feeding tube (Argyle™ PVC feeding tubes, Cardinal Health, Dublin, Ireland) and emptied in the open implants. Mouse-sized open type cell delivery devices were seeded with 300 IEQ and placed in a non-adherent 6-wells plate in 5 mL medium. Rat-sized implants were loaded with 3000 IEQ and placed in a non-adherent, 55 mm petri dish in 10 mL medium.

### Live/Dead Viability Assay

A LIVE/DEAD viability/cytotoxicity kit for mammalian cells (ThermoFisher Scientific) was used according to the manufacturer's instruction to examine the viability of free-floating control rat islets and islets seeded within the open implants at days 1 and 7 of culture. Images were taken using a Nikon Eclipse Ti inverted microscope and analyzed using FIJI software (https://fiji.sc/). Live/dead images were quantified based on work by Spaepen et al., determining cell viability based on the size of the area that was stained for either live or dead staining. Finally, cell viability was calculated according to formula 1.

$$Viability = ( 1 - \frac{{Area}_{dead}}{{Area}_{alive}} ) 100 \% \qquad ( 1 )$$

### Glucose-Stimulated Insulin Secretion (GSIS) Test

Kreb's buffer stock solution (25 mM HEPES, 115 mM NaCl, 24 mM NaHCO₃, 5 mM KCl, 1 mM MgCl₂·6H₂O, 2.5 mM CaCl·2H₂O, 0.2% bovine serum albumin in sterile water) was supplemented with glucose, forming either a high (16.7 mM) or low (1.67 mM) glucose solution. Medium was removed from all samples on days 1 and 7. Samples were washed and incubated for 1 h in low glucose solution at 37° C. to wash out all remaining insulin. Afterwards, all samples were incubated for another 1 h in fresh low glucose solution followed by 1 h of incubation in high glucose solution. The samples were washed 3 times with low glucose solution and incubated for 1 h in low glucose solution. After each incubation step, an aliquot of glucose solution was stored at −30° C. until an insulin ELISA assay was performed. Next, the Kreb's buffer solutions of all samples were replaced for acid ethanol (1.5% HCl in 70% ethanol) and incubated for 5 min. Samples were then transferred to an Eppendorf tube and stored at −30° C. until the ELISA assay. ELISA kits for rat insulin (Mercodia, Uppsala, Sweden) were used to determine the insulin concentration after GSIS according to the manufacturers instruction. The optical density of the samples was read at 450 nm with a spectrophotometric plate reader (CLARIOStar Plus, BMG Labtech). Samples were diluted with Krebs buffer when needed. Finally, the stimulation index (SI) of the pancreatic islets was calculated by dividing the insulin secretion during the high glucose incubation step by insulin secretion during the first low glucose incubation step. Pancreatic islets exhibiting an SI≥2 were considered functional.

### Upscaling

Dimensions for animal implants were calculated with 300, 3000 and 13000 microwells for mouse-, rat- and mini-pig-sized open implants respectively. An aspect ratio of 1 was used for mouse-sized implants (round implant), while 1.5 was used for all other implants (oval implant). All human-sized implants were calculated with an isolation index number (IIN) of 1.5. Three specific cases of human-sized implants holding 200,000 IEQ, 450,000 IEQ or 700,000 IEQ were evaluated, based on the average minimal transplantation IEQ dose of human islets for a 70 kg patient across several clinical centers across Europe.

### Statistics

All results were presented as mean±standard deviation (SD). Statistical analysis were performed using Graphpad PRISM 8. P-values <0.05 were considered statistically significant. Group comparisons were performed using one-way analysis of variance (ANOVA) with Tuckey's post hoc test after assessing the assumptions of equality of variance (Brown-Forsythe test) and normality (Shapiro-Wilk test). If the assumption of normality was not validated, the Kruskal-Wallis test in combination with Dunn's test were used. Direct comparison between two groups was performed by an unpaired t-test after assessing the assumptions of equality of variance and normality. Welch's correction was used for t-tests if the assumption of equality in variance was violated. A Mann-Whitney test was performed if both assumptions of equality of variance and normality were violated.

### Results

### Microwell Thin Films

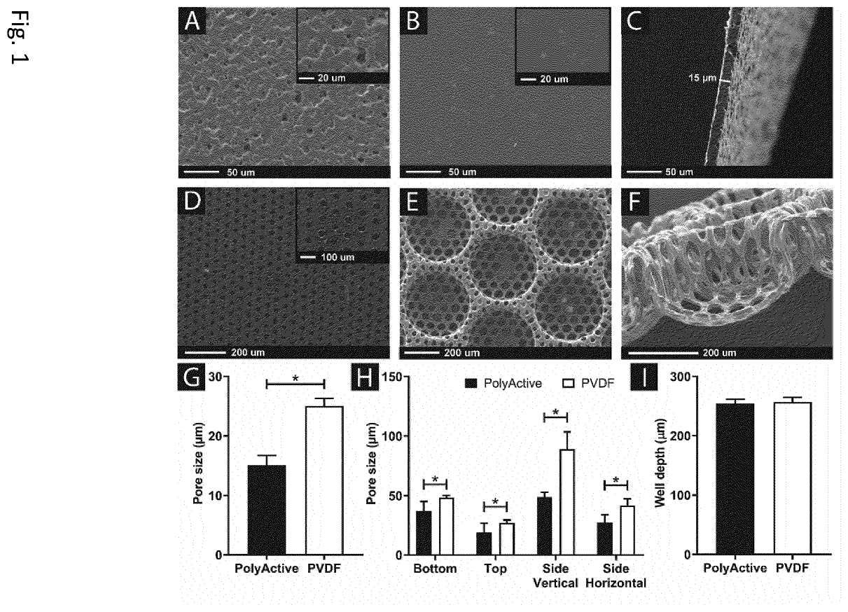

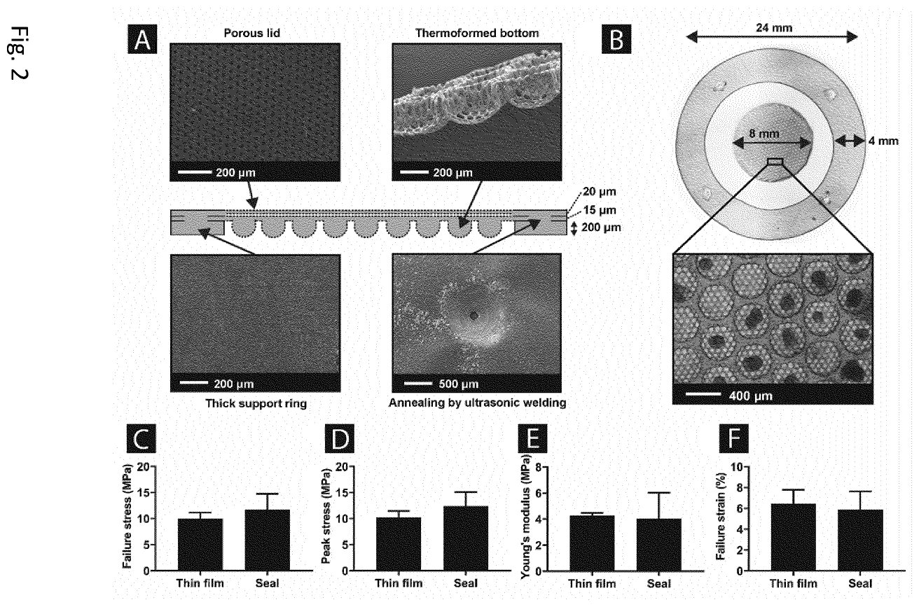

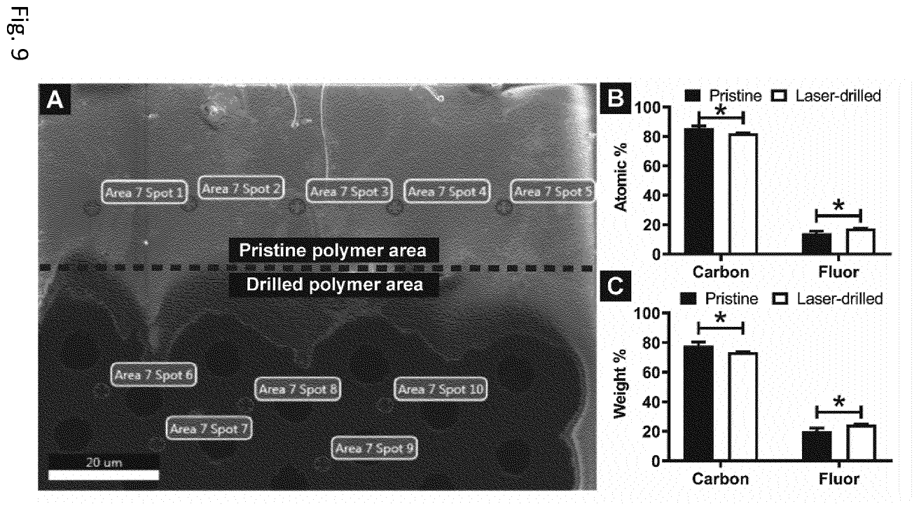

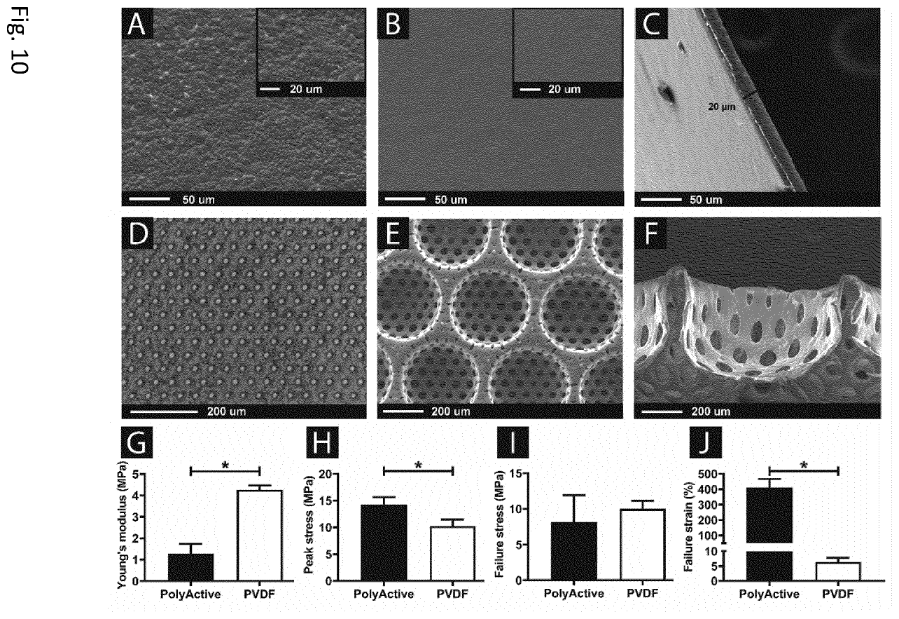

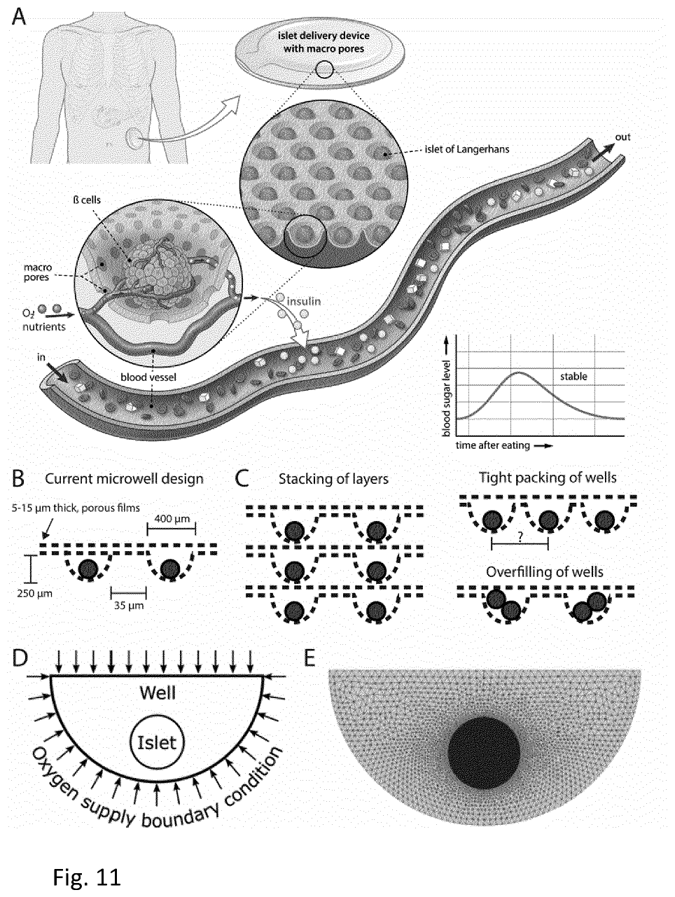

PVDF films were casted with an automatic film caster resulting in 15-20 μm-thick polymer films (FIG. 1 A-C). Next, a predetermined pattern of equally sized pores was created by laser micro-machining (FIG. 1D). Polymer films used for microwell bottom films were patterned with pores having a pore size of 24±1 μm and a pitch of 50 μm, while polymer films used as lids had a pore size of 40±1 μm and pitch of 100 μm (FIG. 1G). Polymer films were darkened after laser micro-machining, but energy dispersive X-ray analysis did not indicate increased Carbon levels indicative of incineration of the polymer (FIG. 9). The laser micro-machined pores were anisotropically stretched during micro-thermoforming. Pores situated at the bottom and top of the well displayed a rounded shape with a pore size of 48±2 μm and 27±2 μm respectively (FIG. 1H). Pores located at the sides of the wells were ellipse-shaped along the depth of the wells, and displayed an average minor diameter of 42±6 μm and major diameter of 89±14 μm. Micro-thermoforming was applied to create microwell structures that displayed an average well diameter of 390±12 μm and well depth of 260±6 μm (FIG. 11). Based on analysis of microwell cross sections, it was estimated that every PVDF microwell holds 55 pores. In general, pore sizes were larger in post micro-thermoforming PVDF films compared to PolyActive films (FIG. 1 G, H, Supplementary FIG. 10). The pores situated on the top of the microwells of PolyActive films displayed the Poisson effect (contraction of the material in a perpendicular direction to the direction of loading), leading to the formation of stretched pores in a 3:1 ratio (FIG. 10E) while pores in PVDF films remained rounded (FIG. 1E).

### Implant Assembly

All PVDF implants were assembled by annealing a 200 μm-thick support ring, microwell-shaped film and porous lid at specific points by ultrasonic welding (FIG. 2A, FIG. 7). Each weld led to a circular seal with a diameter of 0.5 mm including an empty core of 150 μm. Mouse-sized implants were sealed together at 4 spots with a fixed distance of 14 mm between each welding spot. The mouse-sized implant retained an outer diameter of 24 mm, microwell-imprinted area with a diameter of 8 mm holding 300 microwells and a 4 mm-wide support ring (FIG. 2B). The strength of the welded spots was evaluated through mechanical tensile testing and compared to the polymer thin films (FIG. 2 C-F). There were no statistical differences in failure stress, peak stress, Young's modulus, or failure strain between US welded seals and thin films.

### Rat Islet Viability and Functionality in Mouse-Sized Open Type Cell Delivery Device

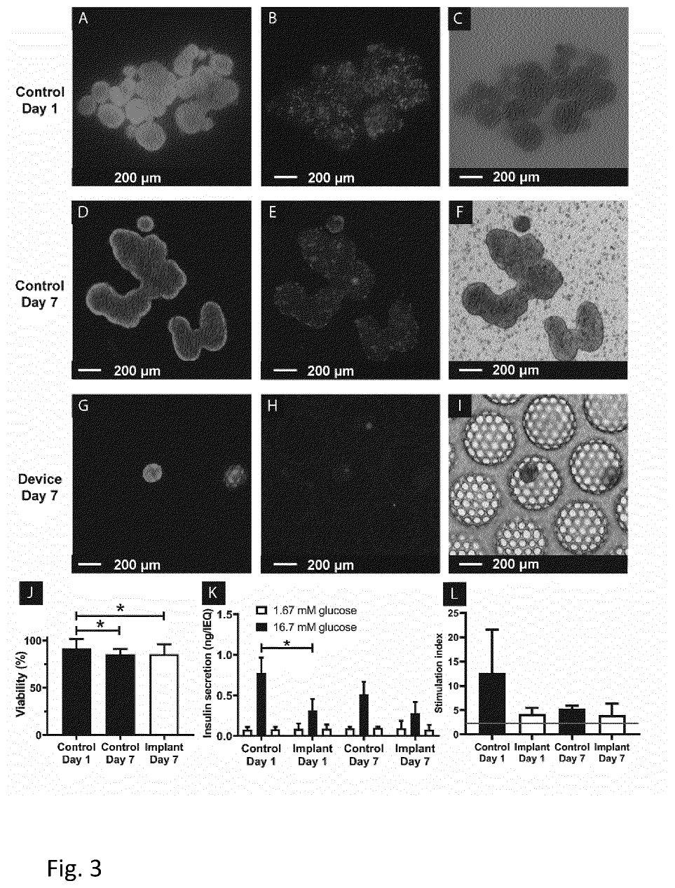

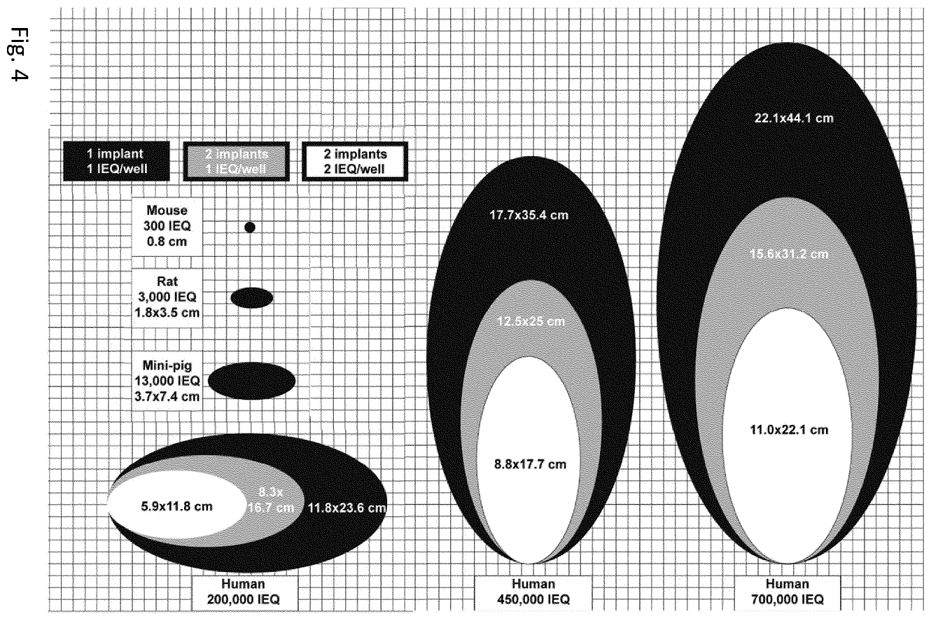

Mouse-sized open type cell delivery devices were loaded with rat islets, after which cell viability was assessed. The pancreatic islets of mice and rats are similar, but isolation yields are considerable higher for rats (100-150 islets for mice and 300-800 islets for rats). On average, 757 pancreatic islets were isolated for each rat, which translated to 1534 IEQ and an islet isolation number (IIN, average number of IEQ/islet) of 2.02. Isolated islets showed a purity >85%. Next, 300 IEQ were seeded within the mouse-sized open type cell delivery device with the help of a cell seeding tool and catheter (FIG. 8). Rat islets seeded within the mouse-sized implant were evenly distributed over the microwells. Some wells were left empty, while double-filled wells were hardly observed. Cell viability was determined at days 1 and 7 (FIG. 3A-1). Notably, individual free-floating control islets aggregated over a 7-day period, while islets seeded in the implant remained separate. Aggregated islets showed a maximum diffusion distance, determined as distance between the center of an islet and the border of the aggregate, of 93±19 μm. Rat islets within the free-floating control group displayed a significantly higher viability at day 1 (92±10%) compared to day 7 control (85±6%) (FIG. 3J). Viability of islets seeded in the implant (86±10%) were similar to control samples at day 7.

Next, rat islets were subjected to a GSIS test to evaluate whether embedding in the microwell implants would affect islet functionality. Control and implant samples both showed the characteristic low-high-low pattern, indicative of normal islet function (FIG. 3K). Control islets displayed a higher insulin secretion during high glucose incubation steps at day 1 (0.78±0.18 ng insulin/IEQ) compared to islets cultured in the implant at day 1 (0.31±0.14 ng insulin/IEQ). This effect was lost after 7 days of culture, when control islets (0.52±0.15 ng insulin/IEQ) displayed similar insulin secretion levels compared to islets in the implant (0.28±0.14 ng insulin/IEQ). In addition, the SI was similar for free-floating control islets (4.0±2.3) and islets seeded in the open type cell delivery device (5.3±0.6) after 7 days of culture (FIG. 3L). Pancreatic islets in both groups and time points were considered functional, as they exceeded the SI≥2 threshold.

### Upscaling

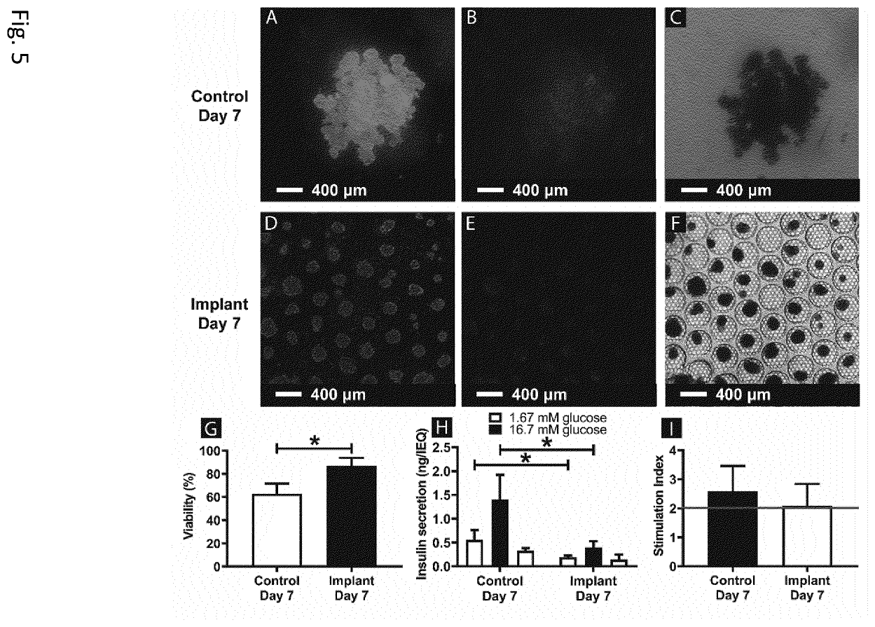

Fictional donor and recipient data (based on real world clinical islet transplantations) were used to calculate the number of required wells. This number was subsequently distributed over the implant's surface depending on implant characteristics such as shape and aspect ratio, finally leading to calculation of implant dimensions, which are given as minor diameter×major diameter. All implant dimensions only include the microwell area and exclude the support ring. Mouse-, rat- and mini-pig-sized implants were designed (IIN=1, well distance=35 μm, amount of implants=1 and IEQ/well=1) requiring a round microwell area with diameter of 0.8 cm holding 300 IEQ or oval microwell areas of 1.8×3.5 cm holding 3000 IEQ and 3.7×7.4 cm holding 13,000 IEQ respectively. Human-sized implants were designed based on the average minimal transplantation IEQ dose utilized by clinical centers across Europe for a 70 kg patient (FIG. 4B). Implant dimensions were calculated (IIN=1.5, aspect ratio=0.5 and well distance=35 μm) for implants holding the lowest (200,000 IEQ), mean (450,000 IEQ) and highest dosage (700,000 IEQ), while varying the amount of implants and IEQ/well for each case. Single human-sized implant dimensions (black shapes) ranged between 11.8×23.6 cm to 22.1×44.1 cm. In the model, implant dimensions were kept small by distributing all islets over two implants (grey shapes), leading to implant dimensions between 8.3×16.7 cm and 11.0×22.1 cm, and were further downsized by seeding two IEQ/well (white shapes), leading to implant sizes varying between 5.9×11.8 and 11.0×22.1 cm.

### Rat Islet Viability and Functionality in Rat-Sized Device

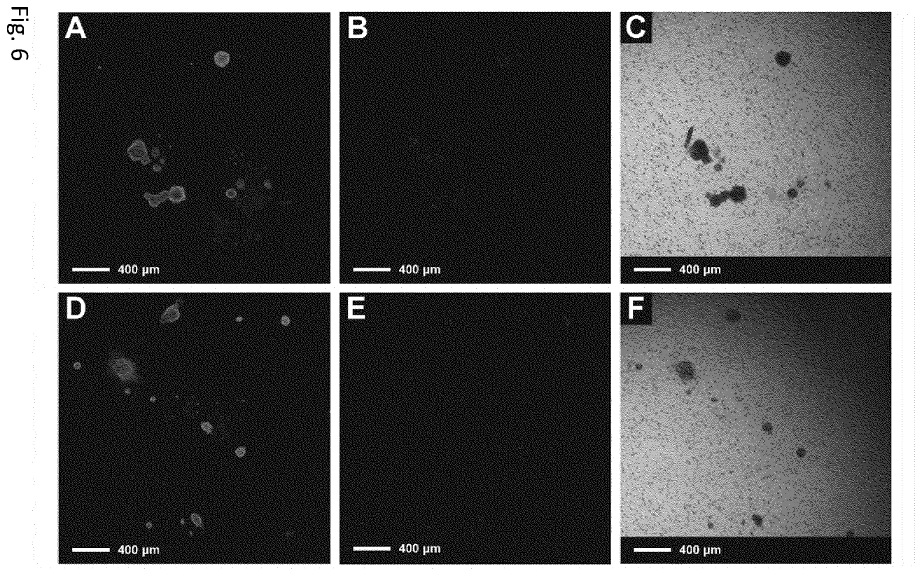

Rat-sized open type cell delivery devices were manufactured with final implant dimensions of 2.6×4.4 cm (FIG. 7F). Implants were US welded according to upscaled version of the welding guide (FIG. 7D), sterilized, clamped in an expanded seeding tool and subsequently seeded with rat islets (FIG. 8 C-E). Free floating controls were seeded with 300 IEQ at 500 IEQ/cm² while implants were seeded with 3000 IEQ at 600 IEQ/cm². Control samples displayed the formation of a large aggregate (diameter of 1200±300 μm), resulting in a necrotic core over 7 day in vitro culture (FIG. 5A-C). Islets seeded within the implants remained separate from one another, only displaying multiple islets in a well case of small islets with a diameter below 100 μm (FIG. 5D-F). The device group showed a significantly higher viability of compared to the control group (87±7% vs 63±9% respectively) (FIG. 5G). Control islets displayed a higher insulin secretion during high glucose incubation steps at day 7 (1.40±0.52 ng insulin/IEQ) compared to islets cultured in the device at day 7 (0.40±0.12 ng insulin/IEQ). In addition, control islets displayed a higher insulin secretion during the first low glucose incubation (0.56±0.20 ng insulin/IEQ VS 0.19±0.04 ng insulin/IEQ). The SI was similar for free-floating control islets (2.6±0.9) and islets seeded in the open type cell delivery device (2.1±0.7) after 7 days of culture (FIG. 3L). Pancreatic islets in both groups and time points were considered functional, as they exceed the SI≥2 threshold.

### Human Islet Viability and Functionality in Rat-Sized Device

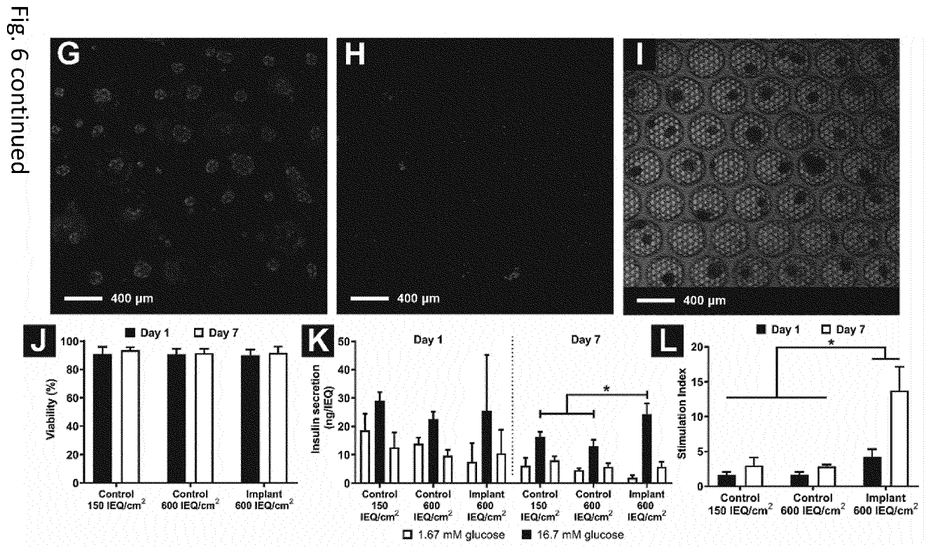

Human islets were seeded in to rat-sized open type cell delivery devices in a similar fashion as was done for rat islets. Controls were seeded with either 90 IEQ (150 IEQ/cm²) or 360 IEQ (600 IEQ/cm²), while devices were seeded with 3000 IEQ (600 IEQ/cm²). Control samples did not show aggregation, but adhered to the culture insert over a 7-day culture period (FIG. 6 A-F). Human islets seeded within the device seemed to adhere to the surface, and microwells occasionally hold more than 1 islet (FIG. 6 G-1). There was no statistical difference in viability between low cell density controls, high cell density controls and devices at either day 1 (91.1±4.7% VS 90.7±3.9% VS 90.0±4.2%) or day 7 (93.6±2.1% VS 91.6±3.1% VS 91.7±4.4%) (FIG. 6 J). Insulin secretion levels were similar for controls and devices at day 1, but insulin secreted during high glucose incubation steps were higher in the device (24.4±3.7 ng insulin/IEQ) compared to the controls (16.3±1.8 ng insulin/IEQ and 13.0±2.2 ng insulin/IEQ) after 7 days of culture (FIG. 6 K). Pancreatic islets in all groups were considered functional (SI≥2). However, islets cultured in the devices showed a higher stimulation index compared to controls at day 1 (SI of 1.7±0.4 VS 1.7±0.4 VS 4.2±1.1) (FIG. 6 I). This effect was even clearer at day 7 (SI of 3.0±1.1 VS 2.8±0.3 VS 13.7±3.4).

### Discussion

As described in the examples, the first step was to manufacture mouse-sized devices from clinically approved PVDF. Casted thin films of PVDF showed a smooth and rough side as a result of phase separation (FIG. 1A.B), in accordance to other literature. Films were subsequently laser micromachined and micro-thermoformed to shape them into microwell structures effectively stretching the film and the pores inside it. By nature, islets hold a spherical morphology with a diameter ranging between 50-400 μm. Pores located at the side of the wells were stretched in anisotropic fashion, with the horizontal pore diameter not exceeding the 50 μm threshold, and thereby preventing loss of islets due to the relatively large vertical pore diameter. In total, most pores sizes were around 30-50 μm, which stimulates revascularization. Micro-thermoforming of PolyActive films led to microwell structures similar to PVDF implants. PolyActive films were laser-drilled with smaller pore size compared to PVDF films (FIG. 1G), due to the difference in mechanical properties of both materials. Especially remarkable is the failure strain of PolyActive, which is more than 60 times higher than that of PVDF. In general, the pore sizes of PVDF implants were larger compared to their PolyActive counterpart. The pores on the top of thermoformed PVDF film remain rounded, while those of PolyActive films were oval shaped with a 3:1 major diameter:minor diameter ratio, similar to previous studies.

The second step was the assembly of the open islet delivery device through bonding of a micro-thermoformed bottom film, a porous top film, and a support ring by ultrasonic welding (FIG. 2A,B). The support ring provided mechanical protection for the microwell structures, prevented folding of the implant and improved handling. Tensile tests showed no difference in mechanical properties of polymer thin films and ultrasonically welded bonds between thin films and support rings (FIG. 2 C-F).

A total of 300 IEQ and 3000 IEQ were seeded into each of the mouse-sized and rat-sized open type cell delivery devices using a seeding tool. Even though low-density culture may be beneficial to islets, the involved high effort and costs, and low practicality are major limitations. As a result, human islets are often cultured at relatively high densities (500-1000 IEQ/mL). In addition, high cell densities are required in the implants to decrease final implant dimensions. To study the effect of cell density, rat islet controls consisted of free-floating islets totaling to 100 IEQ (150 IEQ/cm²) for mouse-sized implants and 300 IEQ (500 IEQ/cm²) for rat-sized implants. Free floating human islet controls were distributed over 2 groups with different seeding densities of 100 IEQ (150 IEQ/cm²) or 360 IEQ (600 IEQ/cm²). Mouse-sized implants were seeded with 300 IEQ and rat-sized implants were seeded with 3000 IEQ.

The islet isolation process leads to the disruption of islet vasculature, making the islets dependent on diffusion from its surrounding to get nutrients and oxygen. Most importantly, the maximum distance of a cell from its nearest capillary rarely exceeds 200 μm and is usually less than 100 μm. It has previously been described that isolated islets undergo apoptosis as a result of hypoxia, disruption of islet matrix, and exposure to cytokines and endotoxins. In addition, central necrosis contributes to cell death in culture and depends on the islet density, the amount of clumping, the size of the islets and the degree of apoptosis during culture.

Rodent control islets seeded at 150 IEQ/cm² showed aggregation over the culture period, leading to the formation of irregularly shaped aggregates with a maximum diffusion distance below 100 um, and maintenance of cell viability (FIG. 3 A-F, J). However, rodent control islets seeded at 500 IEQ/cm² showed formation of a large aggregate with maximum diffusion distances over 500 um (FIG. 5 A-C). A lack of oxygen will result in the formation of necrotic cores and ultimately cell death, explaining the decreased cell viability observed for controls in the 500 IEQ/cm² experiment (FIG. 5 A-G). Similar results were reported in the literature were rodent islets cultured at 600 IEQ/cm² already displayed decreased cell viability after 24 hours of culture compared to islets cultured at 150 IEQ/cm². The remnants of dead cells will trigger the immune system, leading to a more severe immune reaction and an increased risk of implant failure. Control samples holding 3000 IEQ were not taken along, as it was anticipated that cellular functionality and viability would suffer too much due to aggregation of islets, leading to unnecessary animal suffering for islet isolation. In order to prevent aggregation of islets, and subsequent formation of necrotic cores, islets were offered a separate microenvironment through the usage of microwells in the open type cell delivery devices. The microwells effectively separated the islets from each other, leading to a cell viability equal (mouse-implant) or higher (rat-implant) than their respective controls after 7 days of culture (FIG. 3D-J, FIG. 5D-G). Human islets did not aggregate over the 7 day culture period, but seemed to adhere to the surface of the cell culture insert (FIG. 6 A-F). Human islets seeded in the microwell implant (FIG. 6 G-1) displayed a comparable cell viability (>90%) as the control groups (FIG. 6 J). As the human islets adhered to the cell culture insert in the control group, there was no aggregating or fusion of islets, well explaining their maintenance of cell viability (FIG. 6 A-J), but loss of islet viability in the rodent islets. Moreover, the average diameter of rodent islets (100-150 μm) is significantly larger than those of human islets (50-100 um), making them even more prone to central necrosis.

Rodent islets are reported to maintain glucose sensitivity for at least a week in culture, but changes in rodent islet function are known to occur even after a few days. The insulin release data of rodent islets show a similar low-high-low insulin secretion profile and stimulation index (SI≥2) after 7 days of culture for controls and mouse-sized implants, indicative of proper islet functioning (FIG. 3K,L). Given the absence of hypoxia-related necrosis as indicated by the live dead staining, it should come as no surprise that islets in the control and device group behave similarly. However, control rat islets seeded at 500 IEQ/cm2 did show hypoxia-related necrosis, and a significantly higher insulin-release profile compared to islets cultured in the device (FIG. 5H,I). The relatively low insulin release levels from islets in the rat-sized device can be explained by an autocrine feedback loop for insulin release in beta cells, as device samples hold ten times more IEQ compared to control samples. Islets exposed to high insulin levels are therefore believed to secrete less insulin. On the other side, the relative high insulin secretion levels in the control group could be caused due to the necrosis of islets, resulting in the release of intracellular insulin, boosting the released insulin levels during the GSIS test (FIG. 5H). Human islets displayed a similar low-high-low insulin release profile for both controls and implants. Islets cultured in the devices displayed an improved functionality over controls after 7 days of culture, as they secreted more insulin during the high-glucose condition, resulting in a higher stimulation index (FIG. 6 K,L).

Being the native environment of islets, the pancreas is naturally regarded as the most optimal implantation site. Yet it is rarely considered to be used in clinical practice due to the high risk of tissue inflammation (pancreatitis) due to enzyme leakage, and the possible priming of local lymph nodes towards the autoimmune attack on beta cells. The macroencapsulation design of the implant limits the choice of implantation sites due to size constrictions, leaving the peritoneal cavity and subcutaneous space as potential implantation sites. The interperitoneal space offers an interesting implantation site due to its easy accessibility and possibility to house numerous islets. Nevertheless, it is also associated with limited revascularization, delayed glucose responsiveness and chronic hypoxic stress capacity, making it an unattractive site. The subcutaneous space is often considered due to minimal invasiveness, reproducibility and opportunity to easily monitor devices over time or recovery of devices if needed. However, hypoxia and inadequate revascularization are common problems associated with subcutaneous devices, and therefore require prevascularization or other angiogenesis inducing measures such as oxygen generators, growth factors or co-transplantation of mesenchymal stem cells.

We therefore propose a novel implantation site: supramuscular implantation of the islet delivery implant by creating a pocket underneath the muscle fascia of the latissimus dorsi muscle. This novel implantation site should offer a large surface area for implantation, high blood supply to implanted islets and a relative non-invasive surgery. Most notably, the latissimus dorsi muscle is commonly used in reconstructive surgery including head, neck and breast surgery. The latissimus dorsi muscle has a relatively constant anatomy with a large surface area (in some cases even up to 25×40 cm) and is used for tissue grafts >100 cm². Moreover, the muscle can be removed with a relatively easy dissection may problems arise and is known for its minimal donor site morbidity. Removal of the muscle is associated with a reduction in shoulder joint stability, range of motion and strength, but these drawbacks resolve within the next 6-12 months.

The design of the mouse-sized device (holding 300 IEQ) was extrapolated towards rat-sized (holding 3000 IEQ), mini-pig-sized (holding 13,000 IEQ), and several human-sized devices (FIG. 4). An oval shape was chosen for easy implantation. Oval rat-sized devices were subsequently fabricated with outer dimensions of 2.7×4.4 cm, including an oval 1.8×3.5 cm area filled with microwells. (FIG. 7F). Based on the release criteria for islets for clinical islet transplantation across centers in Europe, three different sizes of human implants holding either 200,000, 450,000 or 700,000 IEQ were simulated. Currently, planar implants are made from just one layer, leading to human sized device dimensions up to 22×44 cm, which may be too large for clinical use. We therefore aimed to reduce device dimensions by simulating the distribution of islets over two devices (or double-sided microwell devices), and/or seeding of 2 IEQ/microwell. Nevertheless, the feasibility of multiple layered devices and seeding higher cell densities may also lead to increased local competition for oxygen and nutrients, and should therefore be thoroughly investigated in future studies.

The microwell system can easily be loaded with other cell types than just pancreatic islets. The scarcity of donor tissue is a, if not the most, limiting factor of islet transplantation technology. Lately, several studies have tried to open islet transplantation to a broader audience by in vitro development of pancreatic cells from induced pluripotent stem cells (IPSCs) and embryonic stem cells. Next to the implantation of allogeneic, donor-derived islets, this implant could also easily be used to co-transplant islets with support cells. Possible cell types include mesenchymal stromal cells or endothelial cells, as they have previously shown to improve islet transplantations.

Pancreatic islet delivery devices were manufactured to facilitate extrahepatic islet delivery, aiming to improve clinical islet transplantation. Implants made from clinically approved PVDF showed a similar microwell structure, but improved porosity, compared to previously used PolyActive implants. Ultrasonic welding was used to assemble the implants, which resulted in seals with comparable mechanical properties as PVDF films. Rat and human islets cultured in the microwell-array islet delivery device showed to be viable and functional after 7 day in vitro culture. The mouse-sized device design was extrapolated and upscaled towards rat-, mini-pig-, and human-sized implants with clinically relevant dimensions.

### Example 2

The pancreas is naturally regarded as the most optimal implantation site for islet transplantation. However, the pancreas is rarely considered in clinical practice due to possible priming of local lymph nodes towards the autoimmune attack on β-cells and a high risk of tissue inflammation (pancreatitis) due to enzyme leakage from acinar parts of the pancreas. The islet transplantation field is therefore searching for an alternative extra-hepatic transplantation strategy that stimulates islet survival and functionality.

It is vital to understand the requirements of pancreatic islets in order to develop a successful transplantation strategy. Pancreatic islets are naturally spread over the entire pancreas and make up 1-2% of pancreatic tissue. Islets hold a high metabolic activity and therefore have a relatively high oxygen demand, requiring 15-20% of the pancreatic blood flow. Islets are exposed to a partial oxygen pressure (pO2) of 40-60 mmHg (around 5% O2) within the pancreas, which can increase close to the oxygen tension of arterial blood (80-100 mm Hg) since islets in the pancreas contain a dense capillary network in order to monitor blood glucose levels. During clinical islet transplantation, the pancreas is enzymatically digested to liberate the islets from the acinar tissue by breaking up the extracellular matrix. However, this enzyme cocktail also disrupts the dense capillary network within islets. Therefore, isolated islets solely depend on diffusion of oxygen and nutrients for a period of 7-14 days after transplantation. Nonetheless, even after 3 months, intrahepatic implanted islets show a relative low oxygen tension <10 mmHg, again emphasizing the need for extrahepatic transplantation strategies. Moreover it is known that both glucose responsiveness and insulin secretion of β-cells decrease in hypoxic conditions, leading to a diminished clinical effectiveness. Restoration of oxygen tension upon islet transplantation is therefore crucial to realize desired clinical outcomes.

The porous nature of the device described herein allows swift revascularization of pancreatic islets upon transplantation. Pancreatic islets are distributed over the microwells to prevent islet aggregation, a feature that is unavoidable when transplanting naked cells. Physiologically, the maximum distance of any cell from its nearest capillary rarely exceeds 100-200 μm due to the diffusion limit of oxygen. However, isolated islets have the tendency to aggregate into large cell constructs which form hypoxic cores. This further develops towards a necrotic core if hypoxia is maintained, and finally leads to cell death and diminished functionality. On top of this, the remnants of these dead cells will trigger the immune system, leading to a more severe immune reaction and an increased risk of graft failure. Separating the islets by offering them an individual subspace in the shape of a microwell has been shown herein to improve islet viability and functionality in vitro and in vivo once implanted at the epididymal fat pad. However, predicted device dimensions for microwell devices capable of delivering clinically relevant amount of islets are surgically challenging to implant. Two oval shaped devices should be implanted which both hold device dimensions with minor×major axes of 8×16 cm for 200,000 IEQ up to 16×32 cm for 700,000 IEQ. The islet packing density should therefore be increased in the microwell device to reduce device dimensions. However, too high islet packing densities will realize favourable device dimensions, but will also lead to a severe risk of oxygen and nutrient competition and potentially results in the loss of graft function shortly after implantation. We evaluated three different strategies to optimize the cell packing density in microwell implants: 1) Tight packing of microwells by optimizing the distance in between islets, 2) Overfilling of microwells by multiple islets, 3) Stacking of different layers of microwell devices (FIG. 11 C). As complete in vitro evaluation of these three different strategies is costly and time-consuming, we created a computational model to evaluate the three different strategies in silico.

### Methods

### Fabrication of Microwell Devices

Components of the microwell-array islet delivery devices were manufactured as previously reported (See Example 1). Microwell devices consisted of three different components: (1) a microwell-imprinted, porous film, (2) a planar porous film acting as lid and (3) a support ring. In short, 15 μm-thick films of polyvinylidene fluoride (PVDF or Kynar 720, Solvay) were solvent casted with the aid of an automatic film caster (Elcometer). Films were made porous by laser micro-machining with a UV-short pulse laser at a frequency of 25 kHz. Polymer films used for microwell films were patterned with pores holding pore sizes of 25 μm and 50 μm pitch, while polymer films used as lids were patterned with a pore size of 40 μm and 100 μm pitch. The porous films holding microwells were fabricated through micro-thermoforming at 85° C. and 30 kN in a hydraulic press (Specac), effectively reshaping the planar films into microwell-containing films. The support rings were fabricated by compressing 2 g of PVDF pellets into a 200 μm-thick disc at 180° C. and 20 kN by the same hydraulic press. Support rings were subsequently cut from the 200 μm-thick disc with a cutting plotter (Silhouette Cameo 4). Finally, devices were assembled by an ultrasonic point welding system (manual LPX welding station, Branson) at 75% amplitude for 1 s. Single-layered device were constructed (from bottom to top) as (1) support ring, (2) microwell film, (3) lid, and welded according to a custom-made welding guide to obtain a reproducible pattern of 11 welding spots. Double-layered devices were assembled in a similar fashion, with the exception of the stacking order being (1) support ring bottom layer, (2) microwell film bottom layer, (3) lid bottom layer, (4) support ring acting as spacer, (5) support ring upper layer, (6) microwell film upper layer, (7) lid upper layer.

### Cell Culture

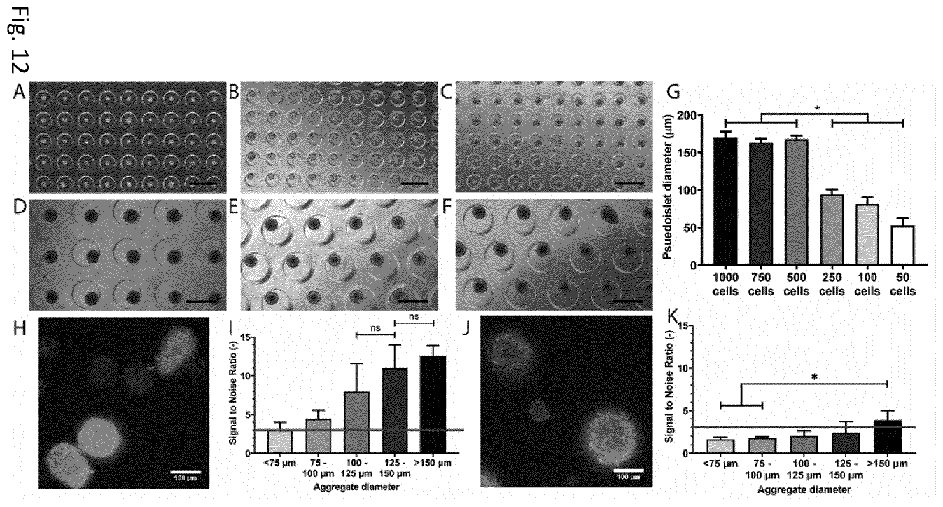

INS1E rat insulinoma β-cells (passage 36-40, Addexbio Technology) were cultured in Roswell Park Memorial Institute (RPMI) 1640 medium with L-glutamine (Sigma Aldrich) supplemented with 10% (v/v) fetal bovine serum (FBS, Sigma), 10 mM HEPES (4-(2-hydroxyethyl)-1-piperazineethanesulfonic acid), 1 mM sodium pyruvate, 5 mM glucose, 23.8 mM sodium bicarbonate and 50 mM beta-mercaptoethanol (all Thermo Fisher Scientific). INS1E cells were aggregated into pseudoislets through a method described by Rivron et al. [42]. In short, a polydimethylsiloxane (PDMS) stamp with 200 μm or 400 μm wide micropillars were placed on the bottom of a 6 wells plate. A heated 3% UltraPure™ agarose (Thermo Fisher Scientific) solution was poured on top of the PDMS stamp, and allowed to cool down and solidify. Agarose discs were then taken out of the 6 wells plate and the PDMS stamp was removed. The agarose disc was then cut to shape and placed in a 12 well plate. Each agarose disc held either 800 microcavities with a diameter of 400 μm or 3200 microcavities with a diameter of 200 μm. A range of differently sized INS1E pseudoislets were aggregated over a three day period by seeding either 1000, 750 or 500 cells in 400 μm microcavities, or 250, 100 or 50 cells in 200 μm wide microcavities, (FIG. 12A-G).

Human islets were provided by the Human Islet Isolation Laboratory at Leiden University Medical Center (LUMC, Leiden, the Netherlands) which has permission from the Dutch government to isolate human islets with clinical intend. Human islets that were not deemed suitable for clinical islet transplantation were used in these experiments, in accordance with Dutch Law. A total of 40.000 IEQ human islets were obtained with a purity of 95%. Islets were cultured in (Connaught Medical Research Laboratories) CMRL-1066 medium (Pan Biotech) supplemented with 10% FBS (Sigma), 10 mM HEPES (Thermo Fisher Scientific), 1% Penicillin-Streptomycin (Thermo Fisher Scientific) and 10 μg/mL ciprofloxacin (Sigma). INS1E cells, pseudoislets and human islets were cultured at 37° C., at either 21% O₂ or 5% CO₂ until the start of experiments. Brightfield images were taken during culture with an Olympus CKX53 microscope equipped with a PLN2X objective. (Pseudo)islets were seeded into islet delivery devices as described previously (See Example 1). In short, a seeding tool was used to clamp the outer border of the device, preventing any cell loss during seeding. A Luer lock syringe was loaded with (pseudo)islets connected to a blunt-tip feeding tube and emptied in the islet delivery devices. Devices were placed in a non-adherent, 55 mm petri dish with 10 mL medium.

### Hypoxia Staining and Imaging

Pseudoislets were harvested from the agarose discs and seeded in a CELLview non-adherent culture dish (glass bottom, 4 compartments, Greiner Bio-One) at a density of 150 pseudoislets/compartment in 0.5 mL of medium. Samples were cultured overnight at normoxia (21% O₂) or hypoxia (5% O₂), but always with 5% CO₂. The free floating human islets were handpicked into three different groups and collected in non-adherent 24 wells plates in 1 mL medium: small (<75 μm), medium (75-150 μm), large (>150 μm) and mixed diameter islets. Islets were subsequently cultured for 2 days under normoxia or hypoxia. On the day of imaging, (pseudo)islets were stained for 1 h with 5 μM Invitrogen™ Image-iT™ green hypoxia dye (Fisher Scientific), after which the medium was replaced for medium with 8 nM Hoechst 33342 (counter stain for cell nuclei, Thermo Fisher Scientific), and incubated at either normoxia or hypoxia for 4 h. The hypoxia dye starts to fluoresce when atmospheric O₂ levels fall below 5% O₂ and the fluorescent signal intensity increases as the O₂ levels decrease further in the environment. Hypoxia imaging was performed on an automated inverted Nikon Ti-E microscope, equipped with a Lumencor Spectra X light source, Photometrics Prime 95B sCMOS camera and an MCL NANO Z500-N TI z-stage. The system was equipped with a CrestOptics X-Light V2 spinning disk unit with a pinhole size of 70 μm. Images were taken with excitation wavelengths 390 nm and 480 nm in combination with DAPI and FITC emission filters, a CFI Plan Fluor DL 10× objective and 2×2 camera binning. Images were analyzed using FIJI software (https://fiji.sc/). The hypoxia staining intensity was quantified over a line profile crossing the (pseudo)islets, including both the islet and the background. The fluorescence intensity of the dye within the (pseudo)islet was then averaged, and was divided by the average fluorescence intensity of the background to calculate the signal to noise ratio (SNR) of the hypoxia staining. The hypoxia threshold was determined as the average SNR of the smallest (pseudo)islet group (<75 μm) cultured in hypoxia.

### Oxygen Imaging

Oxygen sensitive sensor foils (SF-RPSu4, Presens) were glued on the inside of glass bottom petri dishes (12 mm diameter glass bottom, 35 mm petri dish, VWR), cleaned with 70% ethanol and washed three times with cell culture medium. INS1E pseudoislets were seeded within the petri dishes in 1 mL of medium. Local oxygen concentrations surrounding islets were subsequently imaged with a VisiSens oxygen imaging system (Presens) during hypoxia (5% O₂) culture. The system was equipped with an microscope configuration (Presens), leading to a field of view of 2.5×1.8 mm. Prior to the study, the oxygen imaging system was calibrated during a two point calibration in (1) air-saturated (ambient air) and (2) an oxygen-free environment realized by mixing 1 mg/mL sodium sulphite (Na₂SO₃), 50 μL of cobalt nitrate (Co(NO₃)₂ in 0.5 mol/L nitric acid, and 100 mL tap water. Dedicated software (VisiSens ScientifiCal version 1.10) was used to obtain a time series in which images were taken every 5 minutes over 4 h. Subsequently, the software was used to extract oxygen concentrations over line profiles crossing the pseudoislets. Extracted data was averaged with a moving average with an interval of 30 data points.

### Computational Model

In this study, the computational model was developed based on the reaction-diffusion-advection partial differential equation, describing the transport and consumption of oxygen:

$$\frac{\partial c}{\partial t} = ∇ \cdot ( D ∇ c ) - ∇ \cdot ( uc ) + R ( c ) , \qquad ( Equation 1 )$$

in which c=c(x,y,t) is a scalar quantity, D is the diffusion coefficient, and u is an external velocity field. The terms in Equation 1 are corresponding to the temporal evolution of c, the diffusion of it in the domain of interest, its behavior while being advected, and its reaction and consumption patterns, respectively. Since the presence and effect of fluid flow were not taken into account in this work, the simplified form of Equation 1, considering c as the concentration of oxygen, can be written as:

$$\frac{\partial {C}_{{O}_{2}}}{\partial t} = ∇ \cdot ( D ∇ {C}_{{O}_{2}} ) + R ( {C}_{{O}_{2}} ) , \qquad ( Equation 2 )$$

where C_{O}_{₂} is the concentration of oxygen in mol·m⁻³. In order to obtain the final form of the equation, the reaction term was written as a Michaelis-Menten-like equation for the consumption of oxygen [43]:

$$R ( {C}_{{O}_{2}} ) = {R}_{max , {O}_{2}} \frac{{C}_{{O}_{2}}}{{C}_{{O}_{2}} + {C}_{MM , {O}_{2}}} \cdot δ ( {C}_{{O}_{2}} > {C}_{c r} ) , \qquad ( Equation 3 )$$

where R_{max, O}_{₂} is the maximum consumption rate, C_{MM,O}_{₂} is the Michaelis-Menten constant for oxygen concentration, C_{cr} is the critical concentration, and δ is the Heaviside function to cut the consumption where the oxygen concentration falls below the critical concentration. Equation 3 can be subsequently rewritten to include the effect of the metabolic demand of insulin production by considering the local glucose concentration:

$$( Equation 4 )$$

$$R ( {C}_{{O}_{2}} ) = {R}_{max , {O}_{2}} \frac{{C}_{{O}_{2}}}{{C}_{{O}_{2}} + {C}_{MM , {O}_{2}}} \cdot φ \frac{{C}_{gluc}}{{C}_{gluc} + {C}_{MM , gluc}} \cdot δ ( {C}_{{O}_{2}} > {C}_{c r} ) ,$$

in which φ is a constant to tune the effect of glucose, and C_{MM,gluc} is the Michaelis-Menten constant for glucose concentration. Adding Equation 4 to Equation 2 results in the final form of the transport equation used in the current study.

The computational model was implemented by solving the derived equation using the finite element method and the FreeFEM software [44], a domain-specific language for solving partial differential equations. The Picard iterative method was used to handle the non-linearity of the equation in the numerical implementation. The geometry of the wells was modeled as a semi-circle to mimic the shape of wells in the device, and a fixed oxygen supply boundary condition was applied to the well boundaries (FIG. 11 D,E). Table 1 summarizes the selected value of each parameter and coefficient of Equations 2 and 4, as reported in previous studies. A variable diffusion coefficient was used to distinguish the islet (tissue) from the surrounding environment in the well, and the consumption rate was only applied to the islet. The computational mesh was refined on the islet/medium interface to increase the numerical accuracy of the simulations, resulting in ˜7,000 elements for a single islet and ˜230,000 elements for stacking simulations. The simulations were carried out with a long enough time to reach steady-state for single islet simulations. The same time frame was used for stacking simulations to ease data comparison. The model represents islets in a microwell during normoxia cell culture (18.5% O₂) or hypoxia culture (5% O₂, or pO₂ of 40 mmHg simulating islets just after implantation). The model therefore does not include blood vessel ingrowth, and islets solely depend on diffusion of oxygen. In addition, cell death as a result of hypoxia (leading to a decreased oxygen demand) was not included.

TABLE 1
Overview of the parameters of the computational
model including their unit, value and reference(s).

| Parameter | Unit | Value | Reference(s) |
|---|---|---|---|
| D_{O}_{₂} (in medium) | m² · s⁻¹ | 3.0 × 10⁻⁹ | [45] |
| D_{O}_{₂} (in tissue) | m² · s⁻¹ | 2.0 × 10⁻⁹ | [46] |
| R_{max,O}_{₂} | mol · s⁻¹m⁻³ | 0.034 | [47-49] |
| C_{cr} | mol · m⁻³ | 1 × 10⁻⁴ | [47] |
| C_{MM,O}_{₂} | mol · m⁻³ | 1 × 10⁻³ | [43, 49] |
| C_{gluc} | mol · m⁻³ | 11 | [37] |
| φ | — | 3.67 | [37] |
| C_{MM,gluc} | mol · m⁻³ | 8 | [37] |

### Statistics

All results were presented as mean±standard deviation (SD). Statistical analysis were performed using Graphpad PRISM 8. P-values <0.05 were considered statistically significant. Group comparisons were performed using one-way analysis of variance (ANOVA) with Tuckey's post hoc test after assessing the assumptions of equality of variance (Brown-Forsythe test) and normality (Shapiro-Wilk test). If the assumption of normality was not validated, the Kruskal-Wallis test in combination with Dunn's test were used. If the assumption of equal variances was not validated, a Brown-Forsythe and Welch ANOVA test with Dunnett's post hoc test was performed.

### Results

### Hypoxia Imaging of INS1E Pseudoislets

Differently cell numbers of INS1E R-cells were aggregated over a 3 day period, leading to aggregate diameters of 53±9.3 μm for 50 cells, 81±9.1 μm for 100 cells, 95±6.5 μm for 250 cells, 168.3±4.4 μm for 500 cells, 163±5.5 μm for 750 cells and 170±7.8 μm for 1000 cells (FIG. 12 A-G). There was no significant difference in aggregate diameter for cell clusters cultured in the same agarose chips (50, 100 and 250 cells in 200 μm diameter chips and 500, 750 and 1000 cells in 400 μm diameter chips). The degree of hypoxia was assessed with a hypoxia staining after 24 h of culture at hypoxia (5% O₂, FIG. 12 H, I) or normoxia (21% O₂, FIG. 12 J, K). Hypoxia intensity was dependent on pseudoislet diameter, with an average SNR of 3.0±1.0 for <75 μm diameter, 4.4±1.1 for 75-100 μm diameter, 8.0±3.7 for 100-125 μm diameter, 11.0±3.0 for 125-150 μm and 12.6±1.3 for >150 μm diameter pseudoislets cultured in hypoxia. All groups were significantly different from each other, except for the 100-125 μm group VS 125-150 μm group, and the 125-150 μm VS the >150 μm group. The SNR obtained from the smallest pseudoislets cultured under hypoxia was utilized as a hypoxia threshold, meaning that an SNR below this threshold (SNR=3.0) was regarded as background. Pseudoislets cultured under normoxia also showed a SUBSTITUTE SHEET (RULE 26) size-dependent hypoxia intensity, with an average SNR of 1.6±0.3 for <75 μm diameter, 1.8±0.1 for 75-100 μm diameter, 2.0±0.6 for 100-125 μm diameter, 2.4±1.3 for 125-150 μm and 3.9±1.1 for >150 μm diameter pseudoislets. The <75 μm and 75-100 μm group were significantly different from the >150 μm group. Only the >150 μm diameter group crossed the hypoxia threshold, indicating that INS1E pseudoislets <150 μm do not become hypoxic during normoxia cell culture.

### Hypoxia Imaging of Human Islets

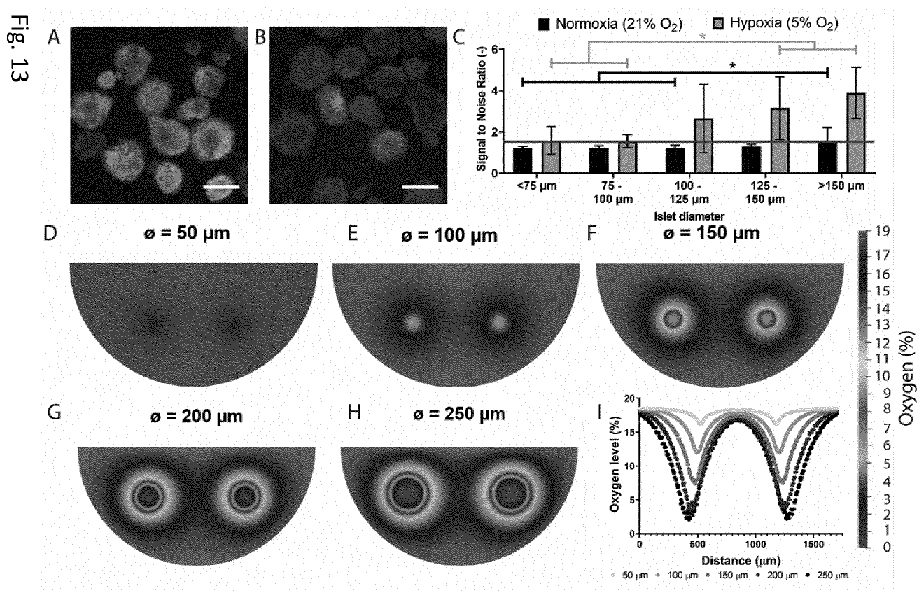

The degree of hypoxia was assessed for human islets with a similar hypoxia staining after 48 h of culture under hypoxia (5% O₂, FIG. 13A) or normoxia (21% O₂, FIG. 13B) and quantified (FIG. 13C). Hypoxia intensity was dependent on pseudoislet diameter, with an average SNR of 1.6±0.6 for <75 μm diameter, 1.6±0.3 for 75-100 μm diameter, 2.6±1.6 for 100-125 μm diameter, 3.2±1.5 for 125-150 μm and 3.9±1.2 for >150 μm diameter islets cultured in hypoxia. The two smallest islet groups (<75 μm and 75-100 μm) were significantly different from the two largest islet groups (125-150 μm and >150 μm). The SNR obtained from the smallest islets was again utilized as hypoxia threshold. Human islets cultured under normoxia also showed a size-dependent hypoxia intensity, with an average SNR of 1.2±0.1 for <75 μm diameter, 1.2±0.1 for 75-100 μm diameter, 1.2±0.1 for 100-125 μm diameter, 1.3±0.1 for 125-150 μm and 1.6±0.6 for >150 μm diameter islets. The <75 μm, 75-100 μm and 100-125 μm groups were significantly different from the >150 μm group. Only the >150 μm diameter group reached the hypoxia threshold, indicating that human islets <150 μm did not become hypoxic during normoxia cell culture. The computational oxygen consumption model was used to evaluate local oxygen levels surrounding islets with diameters between 50 μm and 250 μm under normoxia culture conditions (FIG. 13 D-I). Only islets with diameters >150 μm became hypoxic in their core (16% O₂, 12% O₂, 7% O₂, 3% O₂, 2% O₂ for 50 μm, 100 μm, 150 μm, 200 μm and 250 μm diameter islets respectively), as indicated by oxygen levels below 5% O₂.

### Local Oxygen Imaging of INS1E Pseudoislets During Hypoxia Culture

An oxygen imaging system was utilized to image local oxygen levels surrounding INS1E pseudoislets during hypoxia culture. A time series was collected for 4 h, during which images were taken every 5 minutes. Initially, O₂ levels were high as the incubator door was opened to place the pseudoislets in culture. Background 02 levels then decreased near to 5% O₂ within 10 minutes. Islets were detected as they consume O₂, and therefore decreased their local O₂ levels. After 4 h of culture, differently sized pseudoislets were imaged and local O₂ levels were quantified over a line crossing through the center of the pseudoislets. The core of a relatively small psuedoislet (75 μm diameter) reached 2.9% O₂, while the core of an average sized psuedoislet (125 μm diameter) reached 0.9% O₂ and the core of a relatively large psuedoislet (175 μm diameter) reached 0.0% O₂. The computational oxygen consumption model was used to evaluate local O₂ levels surrounding differently sized islets under hypoxia conditions. Similar to the in vitro experiment, local O₂ levels were quantified over a line crossing through the center of the simulated islets. Oxygen levels within the islet core were predicted to reach 2.7%, 0.2%, 0.1%, 0.1% and 0.1% O₂ for islet diameters of 50 μm, 100 μm, 150 μm, 200 μm and 250 μm respectively.

### Optimal Distance Between Islets

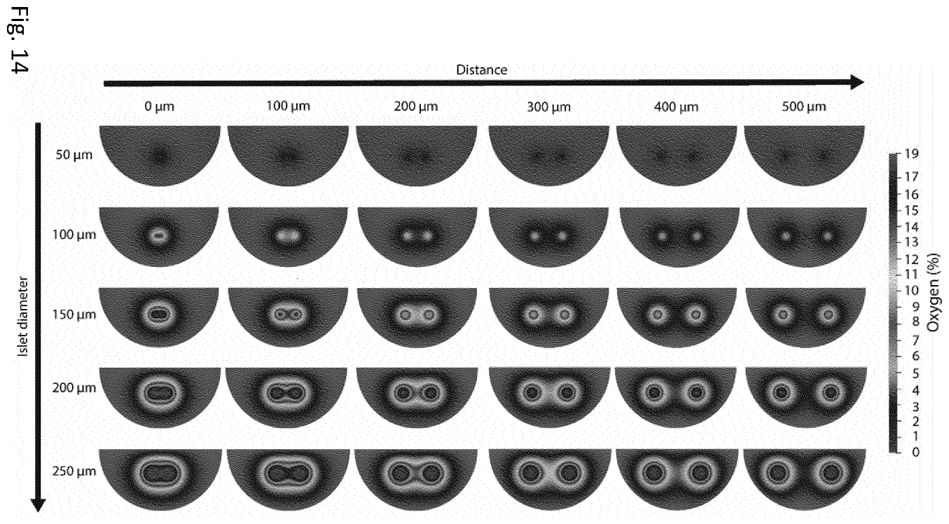

The computational oxygen consumption model was adjusted to simulate two islets. Simulations were run with different islet diameters (50 μm, 100 μm, 150 μm, 200 μm and 250 μm) and distances in between the islets (0 μm, 100 μm, 200 μm, 300 μm, 400 μm and 500 μm) during normoxia culture (FIG. 14). The cores of 200 μm and 250 μm diameter islets became anoxic regardless of the distance in between the islets (<1% O₂ when touching and when 500 μm apart). In all other cases, an islet-islet distance of 500 μm showed no overlap between O₂ consumption areas, and these islets were therefore regarded as two separate islets that did not influence each other. The local O₂ environment of 50 μm were hardly affected when islets were cultured close to each other (predicted O₂ levels in their core of 14% O₂ when touching and 16% O₂ when distanced 500 μm apart). For 100 μm diameter islets, islet core O₂ levels were affected when islets were 0 μm (6% O₂), 100 μm (10% O₂) and 200 um apart (11% O₂), compared to 500 μm apart (12% O₂), but no hypoxic conditions were predicted for any of the islet-islet distances. On the other hand, hypoxia was reached in the cores of 150 μm diameter islets when spaced 0 μm (2% O₂), 100 μm (4% O₂) or 200 μm (5% O₂) apart, but not when islets were spaced 300 μm (6% O₂), 400 μm (6% O₂) or 500 μm (6% O₂) apart.

### Overfilling of Implants

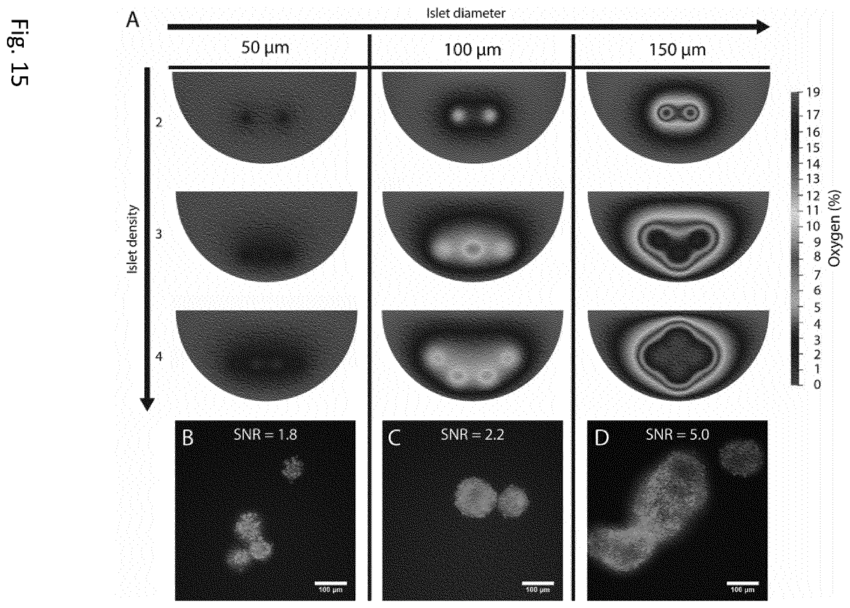

The computation oxygen consumption model was adjusted to simulate two, three or four islets packed within an area similar to a microwell (area of 400 μm wide×250 μm high) (FIG. 15A). Oxygen levels for relatively small islets (diameter 50 μm) were hardly affected by increasing packing densities (16% O₂ for 2 islets, 15% O₂ for 3 islets and 14% O₂ for 4 islets/microwell). Oxygen levels for 100 μm diameter islets were affected more than the smaller islets, with predicted core oxygen levels of 12% O₂ for 2 islets, 11% O₂ for 3 islets and 10% O₂ for 4 islets/microwell. The cores of 150 μm diameter islets became hypoxic in all three packing densities, and core O₂ levels further decrease with increasing packing densities (4% O₂ for 2 islets, 2% O₂ for 3 islets and <1% O₂ for 4 islets/microwell). In addition, the hypoxic area surrounding islets increased with increasing packing densities. Representative images from hypoxia staining of small pseudoislets (diameters between 60-80 μm), average-sized psuedoislets (diameters of 90 μm and 120 μm) and large pseudoislets (diameters of 200 μm and 275 μm) show SNR values of 1.8, 2.2 and 5.0 respectively. Hypoxia was only detected in the largest islet diameter group (crossing the hypoxia threshold of SNR=3.0).

### Stacking of Device Layers

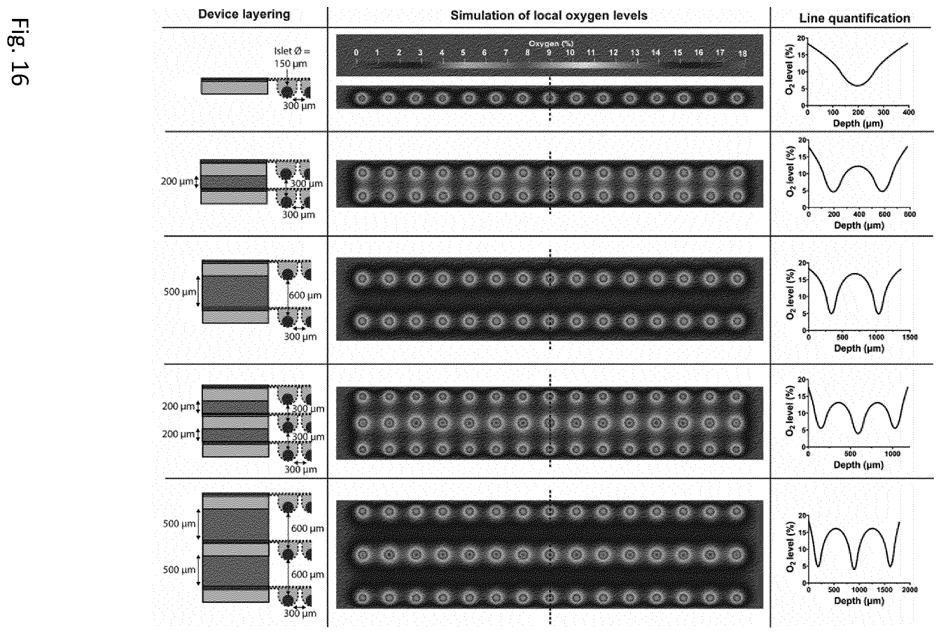

Single layer microwell devices were fabricated which could be seeded with human islets. However, the computational model was adapted to simulate devices consisting of multiple stacked microwell layers. A microwell layer was simulated by a series of fifteen 150 μm diameter islets with each an individual microwell. Device layers were distanced from one another by the use of an extra support layer, which had a thickness of either 200 μm or 500 μm, leading to distance of 300 μm or 600 μm in between islets. Local oxygen levels were quantified over a vertical line profile drawn through the center of the construct. Islets seeded within a single-layered device (250 μm construct thickness) reached core O₂ levels of 6% (FIG. 16, first row). Islets seeded within a double-layered device with either 300 μm interspacing (resulting in a 650 μm thick construct) or 600 μm interspacing (resulting in a 950 μm thick construct) between microwell layers obtained core O₂ levels of 5% (FIG. 16, second and third row). Triple-layered device with either 300 μm interspacing (resulting in a 1150 μm thick construct) or 600 μm interspacing (resulting in a 1650 μm thick construct) between microwell layers obtained core O₂ levels of 5% in the outer microwell layers, but showed a decreased core O₂ level of 4% for islets loaded into the center layer of the 3-layered construct (FIG. 16, fourth and fifth row).

### Manufacturing of Double-Layered Device

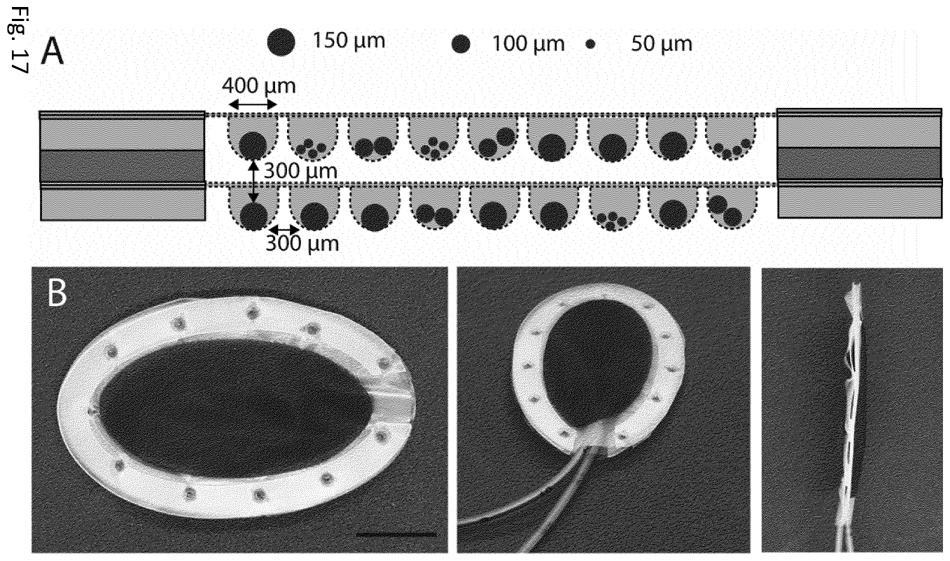

A double-layered microwell construct was manufactured by stacking components of two single-layered devices on top of each other, separated by an extra support ring. The oval shaped device was 26×44 mm in diameters with a total amount of 6000 microwells. The seven different layers of this double-microwell-layered device were manually point welded at 11 separate locations, effectively creating a cell delivery device with two separate pockets holding 3000 microwells, each suitable for cell seeding (FIG. 17B left and middle). All layers were connected one-by-one with a manual point welding system, connecting all seven layers of the construct (FIG. 17B, right).

### Discussion

Proper vascularization is essential to ensure optimal survival and functioning of the transplanted graft. Islets highly depend on diffusion of oxygen and nutrients for the first two weeks after implantation, as transplanted islets are known to revascularize in roughly 14 days [24]. Moreover, oxygen competition results in decreased insulin release and glucose responsiveness [26-28, 50]. It is therefore vital that the design of the islet delivery devices allows a high packing density of islets, without causing too severe competition for oxygen. As a rule of thumb, islet density has been recommended to range between 5-10% of the volume fraction of a macro-encapsulating device [51]. However, this leads to large devices where islets can still cluster together, forming necrotic cores. Therefore, others have tried to increase the islet packing density of macro-encapsulating islet delivery devices through different strategies by enhancing local oxygen supply. The pair device contains islets encapsulated within a flat alginate slab overlain by immunobarriers. The slab was supplied with oxygen through an oxygen-permeable membrane which allowed a gas mixture to reach the encapsulated islets [52, 53]. Others described a PDMS implant with external oxygen supply derived through solvent casting and particle leaching [54]. Unfortunately, this approach requires daily replenishing of the gas mixture through an externalized port. The OxySite device takes another approach in which hydrolytically reactive oxygen-generating biomaterials were incorporated into a PDMS disc [55]. This approach was even enhanced by the incorporation of hemogloblin within the hydrogel carrier, improving oxygen diffusivity through the hydrogel and neutralizing reactive oxygen species, which are harmful side products produced by the oxygen-generating biomaterials [56]. Nevertheless, the long-term durability of oxygen generating biomaterials is still under investigation. We have therefore selected a different strategy by distancing islets from another through a microwell-array device that prevents competition for oxygen and nutrients between islets. The aim of the current study was to optimize device dimensions by fine-tuning the islet packing density within the open microwell implant. Initially, the impact of islet diameter on local oxygen levels was evaluated, followed by the influence of microwell design parameters such as islet-islet distance, overfilling of microwells with multiple islets and layering of microwell layers on local islet oxygen levels.

### Validation of Model by In Vitro Culture of (Pseudo)Islets

The first step was to evaluate the hypoxia levels in differently sized INS1E pseudoislets and human islets during cell culture. INS1E cells were therefore aggregated into pseudoislets over a 3-day period in agarose chips, harvested and subsequently cultured under normoxia or hypoxia. The degree of hypoxia was dependent on (pseudo)islet diameter, with a higher degree of hypoxia in larger aggregates (FIG. 121), which is in overlap with other studies on islet and spheroid culture [21, 37, 57]. Pseudoislets were harvested from the aggregation chips to omit the influence of the microwell cavities in the agarose chips, and provided each (pseudo)islet with a planar base in a petri dish. This however, allowed interactions between (pseudo)islets, which may have influenced their local oxygen levels. The difference in standard deviation in SNR of hypoxia between groups may therefore be a result of oxygen competition between islets during culture. The SNR of the smallest (pseudo)islet group (<75 μm) was set as the hypoxia threshold for each cell type. Interestingly, large aggregates (>150 μm) of both INS1E pseudoislets and human islets crossed the hypoxia threshold when cultured in normoxia conditions. This overlap between pseudoislets and human islets could be explained by the aggregate composition. Pancreatic islets are composed of different cell types; α-cells (30%), B-cells (60%), and γ-, δ- and ε-cells (collectively 10%) [16]. The oxygen consumption rate of α- and β-cells are however similar, allowing the simulation of oxygen consumption of a complete islet solely by focussing on β-cells [21]. Pseudoislets were formed with different cell densities to control the aggregate size over a 3-day period. However, the aggregate size was not significantly different for relatively low seeding densities, as previously also described for INS1E cells [58]. Pseudoislet diameter were similar for 500-1000 cells aggregates, and this difference in cell density may explain the increased standard deviation in SNR in the larger pseudoislets.

The computational O₂ consumption model was used to simulate the local O₂ conditions of pancreatic islets ranging in diameter between 50-250 μm (FIG. 13 D-1). Due to the thin (5-10 μm thick) and porous structure of the microwell device, it was assumed that the device would not affect the diffusion of oxygen towards the human islets. This is supported by work from Lee at al., which showed that solid, 10 μm thick microwells made from low oxygen-permeable PDMS showed an O₂ penetration time of just a few seconds, indicating that thin polymer films hardly influence oxygen permeability [59]. The boundary conditions for in silico modulation of local O₂ levels was set at 18.6% O₂, as in vitro culture of cells under 5% CO₂ will lead to a maximum oxygen concentration of 18.6% when cultured at sea level [60]. The model predicted increasing hypoxia for increasing islet diameters, as was observed in other computational models [26, 37, 38, 41, 57]. In accordance with the hypoxia staining results, hypoxia was reached once islet diameter exceeded 150 μm.

The model was also used to predict local oxygen levels of pseudoislets cultured under hypoxic conditions to simulate the situation when pseudoislets were just implanted in vivo. Local O₂ imaging was used to verify the results obtained from hypoxia staining of pseudoislets. Cell aggregates with diameters of 75 μm, 125 μm or 175 μm were cultured under hypoxia conditions on top of an oxygen-sensitive sensor foil and followed over time. Local O₂ levels surrounding differently sized psuedoislets were quantified over a line profile through the centre of the aggregate, and compared against the local O₂ levels predicted by the model. Again, pseudoislet diameter influenced local O₂ levels, with anoxia conditions (<1% O₂) for aggregates larger than (>100 μm). One should be cautious to compare the exact O₂ levels between the in silico and in vitro results, as the local oxygen levels highly depend on the amount of medium (and therefore the diffusion distance) used [37, 60, 61]. However, the influence of psuedoislet diameter on local O₂ levels was observed both in the model and the O₂ imaging data.

### Optimal Distance Between Islets

Given by the high overlap between the computational model and the in vitro data, the model was deemed verified and used to simulate different packing densities between islets, starting with the distance in between islets. Local O₂ levels were predicted for islets ranging in size (50-250 μm in diameter) and islet-islet distance (0-500 μm) during normoxia culture (FIG. 14). To our knowledge, this is the first paper describing a computational model that predicts the impact of spatial distribution between islets on their local oxygen levels. An islet-islet distance of 500 μm showed no overlap between O₂ environments for any of the islet sizes, and these islets were therefore regarded as two separate islets that did not influence each other. Islets with diameters equal or larger than 200 μm showed cores which became anoxic regardless of islet-islet distance. The local O₂ environment of 50 μm diameter islets were hardly affected by islet-islet distance, most likely due to the limited oxygen consumption of these relatively small aggregates. Core O₂ levels of 100 μm diameter islets were affected when islets were <200 μm apart, but no hypoxic conditions were predicted for any of the islet-islet distances. On the other hand, hypoxia was reached in the cores of 150 μm diameter islets when spaced <300 μm apart. Therefore, an islet-islet distance of 300 μm was regarded as optimal for regular-sized islets.

### Overfilling of Microwells

An increased microwell packing density was evaluated by simulating multiple islets within a single microwell. Local O₂ levels of 50 μm islets were hardly affected even when up to four islets were loaded in a microwell, while 100 μm islets showed a moderate decrease in local oxygen levels (FIG. 15A). Hypoxia staining of psuedoislets with diameters of 50 μm and 100 μm cultured in closed proximity did not reach the hypoxia threshold (FIG. 15B,C). On the other hand, the cores O₂ levels of 150 μm diameter islets were simulated to reach hypoxic conditions with a seeding density of two islets/microwell. This can be related to the optimal islet-islet distance, as the distance between the two islets was below 300 μm. Hypoxia staining confirmed the presence of hypoxia in larger psuedoislets cultured in close proximity (FIG. 15D). Altogether, similarly to the islet-islet distance, the degree of overfilling of microwells depended on the islet size. Cao et al. obtained similar results, and reported that a 300 μm diameter islet encapsulated within a 500 μm-thick alginate capsule showed more severe hypoxia compared to four 100 μm islets in a similar alginate capsule [38].

### Stacking

The most influential upscaling strategy on device dimensions is stacking of multiple microwell layers. The computational model was altered to simulate one, two or three layers with each fifteen islets resembling a single, double- or triple-layered device. Considering the optimal islet-islet distance discussed before, the islets were separated 300 μm from one another within layers. The distance between the layers was varied between 300 μm and 600 μm, as we hypothesized that the increased islet packing density of multiple layers may require a larger distance in between layers to prevent severe O₂ competition between islets. Interestingly, the amount of layers, but not the distance in between layers affected the islet core O₂ levels. Islets simulated at the middle layer of triple-layered devices showed to experience lower O₂ levels compared to the outer layers, indicative of the superiority of double-layered devices over triple-layered devices. Similar results were obtained by Johnson et al. for islets simulated into middle layers of multi-layered alginate slabs [62]. In addition, the diffusion distance between alginate slab-encapsulated islets and their environment has previously been reported to play an important role into local oxygen levels [38]. Double-layered alginate slabs performed better than multi-layered slabs as the diffusion distance was relatively short for both layers, similarly to double-layered devices discussed in this manuscript.

Islets within the middle layer of triple-layered devices with a layer distance of 600 μm were expected to reach more severe hypoxia than triple-layered devices with a layer distance of 300 μm considering their larger diffusion distance, especially taking into consideration that oxygen has a maximum diffusion distance of 200 μm [31, 32]. However, as mentioned in the methodology of the computational model implementation, the time used in all simulations was selected to be equal to the time required for a single islet to reach steady-state. This makes it possible to compare the results of various simulations to one another. However, the stacking model is currently unable to accurately describe the oxygen transport between the wells, as the model was originally developed to only mimic the situation inside a single well with an appropriate oxygen supply boundary condition applied to the surrounding boundaries. If we continue stacking simulations to reach steady-state, all the diffused O₂ will be consumed in the middle layers due to lack of supply. However, keeping the simulation time equal to the steady-state time of a single-well simulation mimicked the condition in which the wells were stacked inside a device. By making this assumption, the simulation results confirmed that the layers far from the O₂ supply boundaries, like the middle layer in the triple-layered device, experienced lower O₂ levels. Improving the model description of the inter-well space and employing longer simulation times may reveal a more pronounced difference in local O₂ levels between the two and triple-layered configurations.

### Clinically Relevant Device Dimensions

We have previously published on a device size calculator which allows the calculation of device dimensions of microwell-array cell delivery devices, based on the desired islet dose and device design [reference to open device paper]. Knowing the optimal islet-islet distance, degree of overfilling and amount of layers for a macro-encapsulating islet delivery device, one can improve the predictions of clinically relevant device dimensions. An important parameter in the device size calculator is the islet isolation index (or islet size index, calculated by IEQ/number of islets, as an indication on the average islet diameter of transplanted islet relative to a 150 μm diameter islet. The islet isolation index of human islet preparations used for CIT have been reported to range between 0.5-2 [33, 63-67]. Therefore, considering an islet isolation number of 1 and slight overfilling with 1.25 IEQ/well, one could transplant 300,000 IEQ distributed over two double-layered devices of 8×16 cm in diameter. Recently, the dimensions of the posterior rectus sheath plane was quantified for over 600 patients, and used to calculate the possible sizes of macro-encapsulating cell delivery devices at this site. Oval-shaped cell delivery devices showed to be superior over rectangular and circular-shaped devices for implantation in the pre-peritoneal space, and could hold an average device with area of 108 cm², equivalent to an oval device with dimensions of 8.3×16.6 cm [68]. It therefore seems that device dimensions of 8×16 cm are reasonable for transplantation at the pre-peritoneal site. Importantly, further decreasing the O₂ competition between islets through other strategies may allow to increase the islet packing density within the device, enabling loading of more islets or reducing device dimensions. The hypoxia experienced by the islets within the center layer of a triple-layered construct may be diminished by oxygen-releasing microbeads, such as utilized within the OxySite device [69]. For instance, device dimensions may be reduced by creating triple-layered devices (two devices with diameters of 6.5×13 cm for 300,000 IEQ) or allow loading of 450,000 IEQ instead of 300,000 IEQ over two triple-layered device of 8×16 cm.

### CONCLUSION

Predicted local oxygen levels surrounding pancreatic islets simulated by a computational model overlapped with hypoxia staining and O₂ imaging of INS1E aggregates and human islets. Local O₂ levels surrounding pancreatic islets were highly dictated by islet diameter. Isolated pancreatic islets which solely depend on diffusion to obtain O₂ become hypoxic (<5% O₂) during normoxia culture (18.6% O₂) if the islets hold a diameter >150 μm. As a result, regularly sized islets (150 μm diameter) should be distanced 300 μm apart to prevent extensive competition for O₂. On the other hand, in a macro-encapsulation strategy where islets can be distributed over microwells, overfilling of the microwells is possible for relatively small islets (s 100 μm in diameter). Double-layered devices still allow sufficient diffusion of O₂ towards the islets, thereby preventing competition for O₂ between layers. On the other hand, triple-layered devices did show increased competition for O₂. Considering these upscaling strategies, upscaled versions of the microwell device design showed to be capable of housing clinically relevant islet numbers with device dimensions suitable for transplantation at the pre-peritoneal site.

1. An open type implantable cell delivery device for transplanting cells in a subject, comprising:
a bottom film having a surface area with a plurality of pores;
a top film having a surface area with a plurality of pores, positioned on top of the bottom film such that the top film substantially covers the bottom film to create an inner space;
wherein the bottom film and the top film are formed from a biocompatible biomaterial, and
wherein the bottom film comprises a plurality of microwells positioned to face the surface area of the top film with the open sides of said microwells,
wherein the pore size of the bottom film and optionally the top film is such that it allows vascularization or vascular ingrowth in the device through the pores.

2. Open type implantable cell delivery device according to claim 1, further comprising:
a supporting structure positioned substantially around the surface area of the bottom film and the surface area of the top film such that the supporting structure is positioned in the plane of the surface areas of the top and the bottom films, wherein the bottom and the top film are attached to the support structure in one or more places such as to leave one or more openings between the top film, the bottom film and the support structures allowing contact between the inner space and the surroundings, preferably wherein the supporting structure is also formed from a biocompatible biomaterial.

3. Open type implantable cell delivery device according to claim 1, wherein the biocompatible biomaterial is selected from polyvinylidene fluoride (PVDF), polycarbonate (PC), polypropylene (PP), poly(ethylene terephthalate (PET), poly(vinyl chloride) (PVC), polyamide (PA), polyethylene (PE), polyimide (PI), polyacrylate, polyolefins, polysulfone (PSF), tetrafluoroethylene/polytetrafluoroethylene (PTFE), ePTFE (expanded polytetrafluoroethylene), polyethersulfone (PES), polycaprolacton (PCL), poly(methyl methacrylate) (PMMA), poly(lactic acid) (PLA) or a combination thereof.

4. Open type implantable cell delivery device according to claim 1, wherein the microwells have a diameter of 200-1000 μm, preferably of 250-950 μm, more preferably of 300-900 μm.

5. Open type implantable cell delivery device according to claim 1, wherein the pore size of the bottom film and optionally the top film is between 5 and 100 μm, preferably between 10 and 80 μm more preferably between 15 and 60 μm most preferably between 20 and 55 μm.

6. Open type implantable cell delivery device according to claim 1, wherein the microwells comprise cells, preferably wherein the cells are organoids or cell clusters, more preferable wherein the cells or cell clusters are or comprise endocrine cells or cytokine producing cells or clusters thereof, more preferably wherein the cells or the cell clusters are selected from islet cells, kidney cells, thyroid cells, thymic cells, testicular cells, pancreatic cells or clusters thereof, or more preferably wherein the organoid is selected from an intestinal organoid, a gastric organoid, a thyroid organoid, a thymic organoid, a testicular organoid, a hepatic organoid, a pancreatic organoid, an epithelial organoid, a lung organoid, a kidney organoid, a gastruloid (embryonic organoid), a blastoid (blastocyst-like organoid), a cardiac organoid, a retinal organoid or a glioblastoma organoid.

7. Open type implantable cell delivery device according to claim 1, wherein the microwells have a diameter of 600-1000 μm, preferably 700-900 μm, more preferably 750-850 μm, optionally wherein the wells comprise organoids or cell clusters which are encapsulated by a hydrogel.

8. Open type implantable cell delivery device according to claim 7, wherein the pore size of the bottom film and optionally the top film is between 5 and 200 μm.

9. Open type implantable cell delivery device according to claim 1, wherein the device comprises a drug infused in or coated on the PVDF of the top film, the bottom film and/or the support structure.

10. Open type implantable cell delivery device according to claim 1, wherein the device comprises two or more stacked versions of the open type implantable cell delivery device as defined in any of the preceding claims stacked on top of each other and optionally separated by a spacer.

11. The open type implantable cell delivery device as defined in claim 1 for use in the treatment, prevention or amelioration of a disease.

12. The open type implantable cell delivery device for use according to claim 11 wherein the treatment is treatment of diabetes, preferably type 1 diabetes.

13. Method of constructing open type implantable cell delivery device, the method comprising:
providing a bottom film having a surface area with a plurality of pores and further comprising a plurality of microwells;
positioning a top film having a surface area, optionally with a plurality of pores, on the bottom film such that the openings of the microwells face the top film, to create an inner space between the bottom and top film in open contact with the microwells;
and optionally, positioning a support structure substantially around the assembly of bottom and top films in the same plane as the films such that the support structure at least partly overlaps with the edges of bottom and the top films;
spot welding the bottom and the top films in two or more places to attach the bottom and top film to each other and/or optionally to the support structure, such as to leave several openings through which the inner space is accessible,
wherein the pore size of the bottom film and optionally the top film is such that it allows vascularization or vascular ingrowth in the device through the pores.

14. Method of seeding an open type implantable cell delivery device, the method comprising:
connecting a container for cells with a first end of a tube, and inserting the second end of the tube through an opening of the open type implantable cell delivery device into the inner space such that the inner space is in open connection with the container;
clamping the exterior of the open type implantable cell delivery device such that all remaining openings are sealed;
loading the container with cells or cell clusters suspended in a suitable medium;
allowing the cells to flow from the container through the tube into the inner space of the open type implantable cell delivery device while excess medium is drained through the pores.

15. Method according to claim 14, wherein the cells are allowed to flow through the tube into the inner space of the open type implantable cell delivery device by gravity.
